SEQUENCE LISTING (26 sequences)

SEQ ID NO: 1 - protein, 690 aa, Artificial Sequence
  Synthetic Construct
EVQLVESGGGLVQPGGSLRLSCAASGFTFTDYTMDWVRKAPGKGLEWVADVNPNSGGSIYNQEFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREQYQSTYRVVSVLTVLHQDWLNGKEYKCKVSNKALPAPIEKTISKAKGQPREPQVSTLPPSREEMTKNQVSLMCLVYGFYPSDIAVEWESNGQPENNYKTTPPVLDSDGSFFLYSVLTVDKSRWQQGNVFSCSVMHEALHNHYTQKSLSLSPGKGGGGSGGGGSGGGGSGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGRGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSC

SEQ ID NO: 2 - protein, 212 aa, Artificial Sequence
  Synthetic Construct
RIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIKGQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTEC

SEQ ID NO: 3 - protein, 691 aa, Artificial Sequence
  Synthetic Construct
QVQLVQSGAEVKKPGASVKVSCKASGYTFTSHWMHWVRYAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPGKGGGGSGGGGSGGGGSGGGGSDIQMTQSPSSLSASVGDRVTITCKSSQSLLYTSSQKNYLAWYQQKPGDAPKLLIYWASTRESGVPSRFSGSGSGTDFTLTISSLQPEDFATYYCQQYYAYPWTFGQGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGEC

SEQ ID NO: 4 - protein, 213 aa, Artificial Sequence
  Synthetic Construct
DIQMTQSPSSLSASVGDRVTITCSASSSVTYMYWYQRKPGKAPKLLIYDTSNLASGVPSRFSGSGSGTDYTFTISSLQPEDIATYYCQQWSSHIFTFGQGTKVEIKRTVAAPSVFIFPPSKEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 684 aa, Artificial Sequence
  Synthetic Construct
QVQLVESGGGVVQPGRSLRLDCKASGITFSNSGMHWVRKAPGKGLEWVAVIWYDGSKRYYADEVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVSTLPPSREEMTKNQVSLMCLVYGFYPSDIAVEWESNGQPENNYKTTPPVLDSDGSFFLYSVLTVDKSRWQQGNVFSCSVMHEALHNHYTQKSLSLSPGKGGGGSGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGRGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSC

SEQ ID NO: 6 - protein, 212 aa, Artificial Sequence
  Synthetic Construct
RIVLTQSPATLSLSPGERATLSCRASQSVSSYLAWYQDKPGQAPRLLIYDASNRATGIPARFSGSGSGTDFTLTISSLEPEDFAVYYCQQSSNWPRTFGQGTKVEIKGQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTEC

SEQ ID NO: 7 - protein, 686 aa, Artificial Sequence
  Synthetic Construct
QVQLQQWGAGLLKPSETLSLTCAVYGGSFSGYYWSWIRYSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPGKGGGGSGGGGSGGGGSGGGGSEIVLTQSPGTLSLSPGERATLSCRASQSVGSSYLAWYQQKPGDAPRLLIYGAFSRATGIPDRFSGSGSGTDFTLTISRLEPEDFAVYYCQQYGSSPWTFGQGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGEC

SEQ ID NO: 8 - protein, 216 aa, Artificial Sequence
  Synthetic Construct
EIVLTQSPATLSLSPGERATLSCRASQSVSSYLAWYQRKPGKAPRLLIYDASNRATGIPARFSGSGSGTDFTLTISSLEPEDFAVYYCQQRSNWPPALTFGGGTKVEIKRTVAAPSVFIFPPSKEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 448 aa, Artificial Sequence
  Synthetic Construct
EVQLVESGGGLVQPGGSLRLSCAASGFTFTDYTMDWVRKAPGKGLEWVADVNPNSGGSIYNQEFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVSTLPPSREEMTKNQVSLMCLVYGFYPSDIAVEWESNGQPENNYKTTPPVLDSDGSFFLYSVLTVDKSRWQQGNVFSCSVMHEALHNHYTQKSLSLSPG

SEQ ID NO: 10 - protein, 222 aa, Artificial Sequence
  Synthetic Construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGRGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSC

SEQ ID NO: 11 - protein, 442 aa, Artificial Sequence
  Synthetic Construct
QVQLVESGGGVVQPGRSLRLDCKASGITFSNSGMHWVRKAPGKGLEWVAVIWYDGSKRYYADEVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVSTLPPSREEMTKNQVSLMCLVYGFYPSDIAVEWESNGQPENNYKTTPPVLDSDGSFFLYSVLTVDKSRWQQGNVFSCSVMHEALHNHYTQKSLSLSPG

SEQ ID NO: 12 - protein, 221 aa, Artificial Sequence
  Synthetic Construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGRGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSC

SEQ ID NO: 13 - protein, 697 aa, Artificial Sequence
  Synthetic Construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGRGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSCGGGGSGGGGSGGGGSGGGGSGGGGSQVQLVQSGAEVKKPGASVKVSCKASGYTFTSHWMHWVRYAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPG

SEQ ID NO: 14 - protein, 220 aa, Artificial Sequence
  Synthetic Construct
DIQMTQSPSSLSASVGDRVTITCKSSQSLLYTSSQKNYLAWYQQKPGDAPKLLIYWASTRESGVPSRFSGSGSGTDFTLTISSLQPEDFATYYCQQYYAYPWTFGQGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGEC

SEQ ID NO: 15 - protein, 696 aa, Artificial Sequence
  Synthetic Construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGRGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSCGGGGSGGGGSGGGGSGGGGSGGGGSQVQLQQWGAGLLKPSETLSLTCAVYGGSFSGYYWSWIRYSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPG

SEQ ID NO: 16 - protein, 215 aa, Artificial Sequence
  Synthetic Construct
EIVLTQSPGTLSLSPGERATLSCRASQSVGSSYLAWYQQKPGDAPRLLIYGAFSRATGIPDRFSGSGSGTDFTLTISRLEPEDFAVYYCQQYGSSPWTFGQGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGEC

SEQ ID NO: 17 - protein, 695 aa, Artificial Sequence
  Synthetic Construct
DIQMTQSPSSLSASVGDRVTITCKSSQSLLYTSSQKNYLAWYQQKPGDAPKLLIYWASTRESGVPSRFSGSGSGTDFTLTISSLQPEDFATYYCQQYYAYPWTFGQGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGECGGGGSGGGGSGGGGSGGGGSGGGGSQVQLVQSGAEVKKPGASVKVSCKASGYTFTSHWMHWVRYAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPG

SEQ ID NO: 18 - protein, 690 aa, Artificial Sequence
  Synthetic Construct
EIVLTQSPGTLSLSPGERATLSCRASQSVGSSYLAWYQQKPGDAPRLLIYGAFSRATGIPDRFSGSGSGTDFTLTISRLEPEDFAVYYCQQYGSSPWTFGQGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGECGGGGSGGGGSGGGGSGGGGSGGGGSQVQLQQWGAGLLKPSETLSLTCAVYGGSFSGYYWSWIRYSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPG

SEQ ID NO: 19 - protein, 706 aa, Artificial Sequence
  Synthetic Construct
EVQLVESGGGLVQPGGSLRLSCAASGFTFTDYTMDWVRKAPGKGLEWVADVNPNSGGSIYNQEFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCGGGGSGGGGSGGGGSQVQLVQSGAEVKKPGASVKVSCKASGYTFTSHWMHWVRYAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGGGGGSGGGGSGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGRGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSCHHHHHHHH

SEQ ID NO: 20 - protein, 699 aa, Artificial Sequence
  Synthetic Construct
QVQLVESGGGVVQPGRSLRLDCKASGITFSNSGMHWVRKAPGKGLEWVAVIWYDGSKRYYADEVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCGGGGSGGGGSGGGGSQVQLQQWGAGLLKPSETLSLTCAVYGGSFSGYYWSWIRYSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGRGTLVTVSSASTKGPSVFPLAPSSKSTSGGTAALGCLVADYFPEPVTVSWNSGALTSGVHTFPAVLQSSGLYSLSSVVTVPSSSLGTQTYICNVNHKPSNTKVDERVEPKSCHHHHHHHH

SEQ ID NO: 21 - protein, 704 aa, Artificial Sequence
  Synthetic Construct
EVQLVESGGGLVQPGGSLRLSCAASGFTFTDYTMDWVRKAPGKGLEWVADVNPNSGGSIYNQEFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCGGGGSGGGGSGGGGSQVQLVQSGAEVKKPGASVKVSCKASGYTFTSHWMHWVRYAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGGGGGSGGGGSGGGGSDIQMTQSPSSLSASVGDRVTITCKSSQSLLYTSSQKNYLAWYQQKPGDAPKLLIYWASTRESGVPSRFSGSGSGTDFTLTISSLQPEDFATYYCQQYYAYPWTFGQGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGECHHHHHHHH

SEQ ID NO: 22 - protein, 693 aa, Artificial Sequence
  Synthetic Construct
QVQLVESGGGVVQPGRSLRLDCKASGITFSNSGMHWVRKAPGKGLEWVAVIWYDGSKRYYADEVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCGGGGSGGGGSGGGGSQVQLQQWGAGLLKPSETLSLTCAVYGGSFSGYYWSWIRYSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGGGGGSGGGGSGGGGSEIVLTQSPGTLSLSPGERATLSCRASQSVGSSYLAWYQQKPGDAPRLLIYGAFSRATGIPDRFSGSGSGTDFTLTISRLEPEDFAVYYCQQYGSSPWTFGQGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGECHHHHHHHH

SEQ ID NO: 23 - protein, 321 aa, Artificial Sequence
  Synthetic Construct
WGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREQYQSTYRVVSVLTVLHQDWLNGKEYKCKVSNKALPAPIEKTISKAKGQPREPQVSTLPPSREEMTKNQVSLMCLVYGFYPSDIAVEWESNGQPENNYKTTPPVLDSDGSFFLYSVLTVDKSRWQQGNVFSCSVM

SEQ ID NO: 24 - protein, 322 aa, Artificial Sequence
  Synthetic Construct
WGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVM

SEQ ID NO: 25 - protein, 105 aa, Artificial Sequence
  Synthetic Construct
GQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTEC

SEQ ID NO: 26 - protein, 107 aa, Artificial Sequence
  Synthetic Construct
RTVAAPSVFIFPPSKEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0132626 A1
(43) Pub. Date: Apr. 25, 2024

(54) Fab-Based Trispecific Antibodies

(71) Applicant: Eli Lilly and Company, Indianapolis, IN (US)

(72) Inventors: Stephen J. DEMAREST, San Diego, CA (US); Xiufeng WU, San Diego, CA (US)

(21) Appl. No.: 17/046,028

(22) PCT Filed: Apr. 10, 2019
(86) PCT No.: PCT/US2019/026708

§ 371 Date: Oct. 8, 2020

Related U.S. Application Data
(60) Provisional application No. 62/657,380, filed on Apr. 13, 2018.

(51) Int. Cl. C07K 16/46; C07K 16/28; C07K 16/32

(52) U.S. Cl. CPC C07K 16/468; C07K 16/2818; C07K 16/2863; C07K 16/2878; C07K 16/32; C07K 2317/31; C07K 2317/526; C07K 2317/55; C07K 2317/64; C07K 2317/92

(57) ABSTRACT

The present invention generally relates to Fab-based antibody-like binding proteins comprising three antigen-binding domains that specifically bind one or more target proteins, wherein the antibody domain interfaces are engineered so that they readily self-assemble. The disclosure also provides methods for making trispecific binding proteins and uses of such binding proteins.

The present disclosure relates to trispecific IgG antibody-like compounds containing engineered antibody heavy chain/heavy chain and antibody light chain/heavy chain domain interfaces to enable improved levels of correct self-assembly.

Monoclonal antibodies (mAbs) are having a profound impact on human health and disease management. Over 50 mAbs are FDA approved and 100s are in clinical trials (Ecker, et al., 2015, Reichert 2017). However, typically mAbs only recognize a single antigen, which places a limitation on the extent to which they can intervene in diseases such as cancer and autoimmunity that emerge from complex and multifaceted molecular and cellular dysregulation. Bispecific antibodies (BsAbs) have been the next step to achieve improved efficacy. BsAbs enable combination therapy in a single molecule, and also provide the capacity for enhanced cell specificity (Mazor, et al., 2015, Mazor, et al., 2017), receptor crosslinking (Weidle, et al., 2014), immune cell redirected tumor killing (Huehls, et al., 2015) and many other complex mechanisms to intervene in disease. Methodologies for generating stable, manufacturable BsAbs with mAb-like pharmacokinetics and potential immune cell recruitment through the Fc have significantly advanced in recent years (Brinkmann and Kontermann, 2017). These novel BsAb represent a next wave of therapeutics with the goal of achieving improved efficacy with over 50 moieties currently in clinical development (Brinkmann and Kontermann 2017).

Going beyond BsAb binding to engage more than two antigens, is also desirable. For example, antibodies that effectively engage more than two antigens (e.g., trispecific antibodies) may be developed to provide enhanced specific tumor targeting using multiple tumor associated antigens while still engaging immune cells for redirected lysis. Under selective pressure via the targeting of a single growth and/or survival pathway, tumor cells may switch to one or more additional and redundant pathways to thrive (Van Emburgh, et al., 2014, Jonas, et al., 2016, Kjaer, et al., 2016). A prime example is the co-expression or upregulated expression of HER-2 or cMet receptor tyrosine kinases in head and neck cancer in response to EGFR mAb therapy (Burtness, et al., 2013). Targeting three receptors simultaneously may more effectively repress tumor escape or survival. Similarly, checkpoint inhibitors have become a new effective paradigm for immunogenic cancer types having demonstrated long-term remissions or cures in a modest subset 10-20% of melanoma and non-small cell lung cancer patients. These responses are sometimes improved by combination of checkpoint inhibitors/agonists, albeit with concomitant increases in autoimmune side-effects (Larkin, et al., 2015, Dempke, et al., 2017). For example, combinations of mAbs directed to PD-1, CTLA-4, and CD137 with or without anti-CD19 have the ability to ablate multiple syngeneic tumors in mice (Dai, et al., 2015). Multi-modal targeted therapy in the autoimmune setting also holds promise; however, clinical combinations and bispecifics in this space are still in their infancy. In infectious diseases, targeting multiple antigens or epitopes can lead to improved efficacy as has been demonstrated for HIV, influenza, and even Ebola (Chen, et al., 2013, Dixit, et al., 2016, Madelain, et al., 2016). In all these cases, an antibody-like molecule with the ability to engage three or more epitopes or antigens has the potential to achieve superior efficacy.

Developing therapeutics with the ability to bind three or more antigens significantly raises the complexity of these proteins. Trispecific antibodies (TsAbs) have been described that use multiple antibody variable domain fragments (Castoldi, et al., 2012, Dimasi, et al., 2015, Schmohl, et al., 2016, Egan, et al., 2017). Antibody fragments such as a single chain Fv (scFv) can have stability and solubility limitations, and when using multiple fragments, these limitations can be compounded and require significant engineering (Demarest and Glaser 2008). Another major challenge with utilizing full antibody Fab moieties as building blocks for BsAbs is achieving correct heavy chain (HC)/light chain (LC) pairing when co-expressing multiple heavy and light chains simultaneously. This has remained a challenge even for BsAbs (Klein, et al., 2012), which is compounded in TsAbs that comprise three unique Fab moieties. Therefore, there remains a need for alternative methods for generating TsAbs

Accordingly, we describe herein trispecific binding proteins that use native-like antibody Fab domains, as opposed to antibody Fv, domain antibody, or alternative scaffold proteins, and known interface designs to induce correct chain pairing within the TsAbs (see, e.g., Lewis, et al., 2014, Leaver-Fay, et al., 2016, Froning, et al., 2017; and US Patent Application Publication US20160039947). Given the very low theoretical assembly levels expected in the absence of interface designs (<2% for the IgG Fc-containing TsAbs), the level of correct assembly achieved within the TsAbs formats disclosed herein is extraordinarily high. Surprisingly, half of the TsAbs demonstrated >80% correct HC/LC and HC/HC assembly with a few >90%. This level of correct assembly achieved with the interface designs of the present invention facilitate the generation of TsAbs without complex post-expression purification schemes.

Thus, the present invention provides TsAbs and methods for producing them recombinantly in mammalian cell expression systems.

In some embodiments the binding proteins described herein comprise:
a. a first heavy chain (HC1), comprising, in order from the N-terminus to the C-terminus, (i) a first heavy chain variable region (VH) comprising a lysine at residue 39 and/or a glutamate at residue 62, (ii) a first CH1 domain comprising an alanine at residue 172 and/or a glycine at residue 174, (iii) a CH2 domain, and (iv) a CH3 domain;
b. a first light chain (LC1), comprising, in order from the N-terminus to the C-terminus, (i) a first light chain variable region (VL) comprising an arginine at residue 1 or an aspartate at residue 38, (ii) a first light chain constant region comprising a tyrosine at residue 135 or a tryptophan at residue 176;
c. a second heavy chain (HC2), comprising, in order from the N-terminus to the C-terminus, (i) a second V_{H} comprising a tyrosine at residue 39, (ii) a second CH1 domain comprising a cysteine at residue 127, an aspartate at residue 228 or a serine at residue 230, (iii) a CH2 domain, and (iv) a CH3 domain;
d. a second light chain (LC2), comprising, in order from the N-terminus to the C-terminus, a second VL comprising a arginine at residue 38, (ii) a light chain constant region comprising a lysine at residue 122; and
e. an antigen-binding Fab comprising (i) a third VH comprising an arginine at residue 105, (ii) a CH1 domain comprising an alanine at residue 145 and a glutamate residue at 221; (iii) a VL comprising an aspartate at residue 42, and (iv) a light chain constant region comprising a lysine at residue 123 and an arginine at position 131, wherein i) the N-terminus of the VH of the Fab is linked by a peptide linker to the C-terminus of the CH3 domain of the HC1 and/or the N-terminus of the VL of the Fab fragment is linked by a peptide linker to the C-terminus of the C_{H}3 domain of the HC2, ii) the N-terminus of the VH of the Fab is linked by a peptide linker to the C-terminus of the CH3 domain of the HC1, (ii) the N-terminus of the VL of the Fab is linked by a peptide linker to the C-terminus of the CH3 domain of the HC2, or (iii) the C-terminus of the CH1 domain of the Fab is linked to the N-terminus of the VH domain of the first or second VH; wherein said first VH and said first VL form a first antigen binding domain, said VH of HC2 and said VL of LC2 form a second antigen binding domain; and said first and second antigen binding domains and the Fab fragment specifically bind one or more target proteins.

In some embodiments the binding proteins described herein comprise:
a. a first heavy chain (HC1), comprising, in order from the N-terminus to the C-terminus, (i) a first heavy chain variable region (VH) comprising a lysine at residue 39 and/or a glutamate at residue 62, (ii) a first CH1/hinge domain comprising an alanine at residue 172 and/or a glycine at residue 174, (iii) a CH2 domain, and (iv) a CH3 domain;
b. a first light chain (LC1), comprising, in order from the N-terminus to the C-terminus, (i) a first light chain variable region (VL) comprising an arginine at residue 1 or an aspartate at residue 38, (ii) a first light chain constant region comprising a tyrosine at residue 135 or a tryptophan at residue 176;
c. a second heavy chain (HC2), comprising, in order from the N-terminus to the C-terminus, (i) a second VH comprising a tyrosine at residue 39, (ii) a second C_{H}1/hinge domain comprising a cysteine at residue 127, an aspartate at residue 228 or a serine at residue 230, (iii) a CH2 domain, and (iv) a CH3 domain;
d. a second light chain (LC2), comprising, in order from the N-terminus to the C-terminus, a second VL comprising a arginine at residue 38, (ii) a light chain constant region comprising a lysine at residue 122; and
e. an antigen-binding Fab comprising (i) a third VH comprising an arginine at residue 105, (ii) a CH1/upper hinge (for example, about 5 amino acids) domain comprising an alanine at residue 145 and a glutamate residue at 221; (iii) a VL comprising an aspartate at residue 42, and (iv) a light chain constant region comprising a lysine at residue 123 and an arginine at position 131, wherein i) the N-terminus of the VH of the Fab is linked by a peptide linker to the C-terminus of the C_{H}3 domain of the HC1 and/or the N-terminus of the VL of the Fab fragment is linked by a peptide linker to the C-terminus of the CH3 domain of the HC2, ii) the N-terminus of the VH of the Fab is linked by a peptide linker to the C-terminus of the C_{H}3 domain of the HC1, (ii) the N-terminus of the VL of the Fab is linked by a peptide linker to the C-terminus of the C_{H}3 domain of the HC2, or (iii) the C-terminus of the C_{H}1/upper hinge (for example, about 5 amino acids) domain of the Fab is linked to the N-terminus of the VH domain of the first or second V_{H}; wherein said first VH and said first VL form a first antigen binding domain, said VH of HC2 and said VL of LC2 form a second antigen binding domain; and said first and second antigen binding domains and the Fab fragment specifically bind one or more target proteins.

In some embodiments, the binding proteins described herein comprise:
a. a first heavy chain (HC1), comprising, in order from the N-terminus to the C-terminus, (i) a first heavy chain variable region (VH) comprising a lysine at residue 39 and/or a glutamate at residue 62, (ii) a first CH1 domain comprising an alanine at residue 172 and/or a glycine at residue 174, (iii) a CH2 domain, and (iv) a CH3 domain comprising a serine at residue 349, a methionine at residue 366, a tyrosine at residue 370 and a valine at residue 409;
b. a first light chain (LC1), comprising, in order from the N-terminus to the C-terminus, (i) a first light chain variable region (VL) comprising an arginine at residue 1 or an aspartate at residue 38, (ii) a first light chain constant region comprising a tyrosine at residue 135 or a tryptophan at residue 176;
c. a second heavy chain (HC2), comprising, in order from the N-terminus to the C-terminus, (i) a second VH comprising a tyrosine at residue 39, (ii) a second C_{H}1 domain comprising a cysteine at residue 127, an aspartate at residue 228 or a serine at residue 230, (iii) a CH2 domain, and (iv) a CH3 domain comprising a glycine at residue 356, a glycine at residue 357, a glutamine at residue 364 and an alanine at residue 407;
d. a second light chain (LC2), comprising, in order from the N-terminus to the C-terminus, a second VL comprising a arginine at residue 38, (ii) a light chain constant region comprising a lysine at residue 122; and
e. an antigen-binding Fab fragment comprising (i) a third VH comprising an arginine at residue 105, (ii) a CH1 domain comprising an alanine at residue 145 and a glutamate residue at 221; (iii) a VL comprising an aspartate at residue 42, and (iv) a light chain constant region comprising a lysine at residue 123 and an arginine at position 131, wherein i) the N-terminus of the VH of the Fab is linked by a peptide linker to the C-terminus of the CH3 domain of the HC1 and/or the N-terminus of the VL of the Fab fragment is linked by a peptide linker to the C-terminus of the CH3 domain of the HC2, ii) the N-terminus of the VH of the Fab is linked by a peptide linker to the C-terminus of the CH3 domain of the HC1, (ii) the N-terminus of the VL of the Fab is linked by a peptide linker to the C-terminus of the CH3 domain of the HC2, or (iii) the C-terminus of the third Fab CH1 domain or the third Fab C_{L} domain of the Fab is linked to the N-terminus of the VH domain of the first or second V_{H}; wherein said first VH and said first VL form a first antigen binding domain, said VH of HC2 and said VL of LC2 form a second antigen binding domain; and said first and second antigen binding domains and the Fab fragment specifically bind one or more target proteins.

In some embodiments, the binding proteins described herein comprise a polypeptide heavy chain (HC), a first polypeptide light chain (LC1), a second polypeptide light chain (LC2), and a third polypeptide light chain (LC3), in which
a. the HC has the formula, in order from the N-terminus to the C-terminus, of VH1-CH1a-L1-VH2-CH1b-L2-VH3-CH1c, wherein VH1, VH2, and VH3 is a first, second, and third heavy chain variable region, respectively, and CH1a comprises an alanine and a glycine at amino acid residues 172 and 174, respectively, CH1b comprises a cysteine, an aspartate, and a serine at amino acid residues 127, 228, and 230, respecitvely, and CH1c comprises an alanine and a glutamate at amino acid residues 145 and 221, respectively;
b. the LC1 has the formula VL1-CLa which associates with the VH1-CH1a region of the HC to form a binding site for a first binding partner;
c. the LC2 has the formula VL2-CLb which associates with the VH2-CH1b region of the HC to form a binding site for a second binding partner;
d. the LC3 has the formula VL3-CLc which associates with the VH3-CH1c region of the HC to form a binding site for a first binding partner, wherein CLa comprises a tyrosine and a tryptophan at amino acid residues 135 and 176 respectively, CLb comprises a lysine at amino acid residue 122, and CLc comprises a lysine and an arginine at amino acid residues, 123 and 131, respectively;
e. L1 and L2 are peptide linkers and
f. each of the CH1 domains contains about 5 amino acids of the upper hinge.

In some embodiments, the binding proteins described herein comprise a polypeptide heavy chain (HC), a first polypeptide light chain (LC1), a second polypeptide light chain (LC2), and a third polypeptide light chain (LC3), in which
a. the HC has the formula, in order from the N-terminus to the C-terminus, of VH1-CH1a-L1-VH2-CH1b-L2-VL3-CLc, wherein VH1 and VH2, is a first and second heavy chain variable region (VH), respectively, and VL3 is a light chain variable region (VL);
b. CH1a comprises an alanine and a glycine at amino acid residues 172 and 174, respectively;
c. CH1b comprises a cysteine, an aspartate, and a serine at amino acid residues 127, 228, and 230, respectively;
d. CLc comprises a lysine and an arginine at amino acid residues, 123 and 131, respectively;
e. the LC1 has the formula VL1-CLa which associates with the VH1-CH1a region of the HC to form a binding site for a first binding partner;
f. the LC2 has the formula VL2-CLb which associates with the VH2-CHb region of the HC to form a binding site for a second binding partner;
g. the LC3 has the formula VH3-CHc which associates with the VL3-CLc region of the HC to form a binding site for a third binding partner;
h. L1 and L2 are peptide linkers; and
i. each of the CH1 domains contains about 5 amino acids of the upper hinge.

Also provided by the present invention are methods for producing a trispecific binding protein described herein comprising:
a. expressing in a host cell a nucleic acid encoding a trispecific binding protein described herein;
b. cultivating said host cell under conditions such that said the trispecific binding protein is produced; and
c. recovering the trispecific binding protein from said host cell.
   In some embodiments of the aforementioned methods the host cell is a mammalian cell, preferably, a HEK293 or CHO cell.

Further, the present invention provides any of the aforementioned binding proteins, wherein each of said three light chain variable domains is human kappa isotype.

The present invention further provides an IgG trispecific antibody produced according to any one of the processes of the present invention. In addition to the preparation of tandem Fab and IgG-Fab TsAbs, the methods described herein may also be employed in the preparation of other higher order multi-valent antigen binding compounds (e.g., tetra-, penta-specific antibodies).

The present invention further provides amino acid sequences encoding the heavy chains and the light chains of the tandem Fabs or IgG-Fab TsAbs of the present invention. In addition, the present invention also provides vectors having nucleic acid sequences encoding the heavy chains and the light chains of any of the Fabs or IgG TsAbs of the present invention. Further still, the present invention provides host cells comprising nucleic acid sequences the encoding one or more of the heavy chain and/or the light chain polypeptides of any of the IgG-Fab TsAbs or tandem Fab TsAbs of the present invention.

### BRIEF DESCRIPTION OF FIGURES

FIGS. 1-6 provides schematic diagrams of trispecific antibodies prepared using the structural designs, methods or procedures of the present invention. For each one of FIGS. 1-6 (A) shows a trispecific antibody having 3 Fabs, one which binds to HER-2, one which binds to EGFR, and one which binds to cMet. For each one of FIGS. 1-6 (B) shows a antibody having 3 Fabs, one which binds to PD-1, one which binds to CD137, and one which binds to CTLA-4. FIGS. 1-6 provide a schematic diagram of various exemplary TsAbs with all three V_{H}/VL and CH1/CL interfaces designed to improve specific HC/LC pairing and, for those TsAbs containing an Fc, C_{H}3 interfaces designed to induce Fc heterodimerization.

The general structure of an “IgG antibody” is very well-known. A wild type (WT) antibody of the IgG type is hetero-tetramer of four polypeptide chains (two identical “heavy” chains and two identical “light” chains) that are cross-linked via intra-and inter-chain disulfide bonds. Each heavy chain (HC) is comprised of an N-terminal heavy chain variable region (“V_{H}”) and a heavy chain constant region. The heavy chain constant region is comprised of three domains (C_{H}1, C_{H}2, and C_{H}3) as well as a hinge region (“hinge”) between the C_{H}1 and C_{H}2 domains. Each light chain (LC) is comprised of an N-terminal light chain variable region (“V_{L}”) and a light chain constant region (“C_{L}”). The V_{L} and C_{L} regions may be of the kappa (“κ”) or lambda (“λ”) isotypes (“Cκ” or “Cλ”, respectively). Each heavy chain associates with one light chain via interfaces between the heavy chain and light chain variable domains (the V_{H}/V_{L} interface) and the heavy chain constant C_{H}1 and light chain constant domains (the C_{H}1/C_{L} interface). The association between each of the V_{H}-C_{H}1 and V_{L}-C_{L} segments forms two identical antigen-binding fragments (Fabs) which direct antibody binding to the same antigen or antigenic determinant. Each heavy chain associates with the other heavy chain via interfaces between the hinge-C_{H}2-C_{H}3 segments of each heavy chain, with the association between the two C_{H}2-C_{H}3 segments forming the Fc region of the antibody. Together, each Fab and the Fc form the characteristic “Y-shaped” architecture of IgG antibodies, with each Fab representing the “arms” of the “Y.” IgG antibodies can be further divided into subtypes, e.g., IgG1, IgG2, IgG3, and IgG4 which differ by the length of the hinge regions, the number and location of inter- and intra-chain disulfide bonds and the amino acid sequences of the respective HC constant regions.

The variable regions of each heavy chain-light chain pair associate to form binding sites. The heavy chain variable region (V_{H}) and the light chain variable region (V_{L}) can be subdivided into regions of hypervariability, termed complementarity determining regions (“CDRs”), interspersed with regions that are more conserved, termed framework regions (“FR”). Each V_{H} and V_{L} is composed of three CDRs and four FRs, arranged from amino-terminus to carboxy-terminus in the following order: FR1, CDR1, FR2, CDR2, FR3, CDR3, FR4. CDRs of the heavy chain may be referred to as “CDRH1, CDRH2, and CDRH3” and the 3 CDRs of the light chain may be referred to as “CDRL1, CDRL2 and CDRL3.” The FRs of the heavy chain may be referred to as HFR1, HFR2, HFR3 and HFR4 whereas the FRs of the light chain may be referred to as LFR1, LFR2, LFR3 and LFR4. The CDRs contain most of the residues which form specific interactions with the antigen. As used herein, “antigen” or “antigenic determinant” refers to a target protein to which an IgG antibody binds, or to a particular epitope (on a target protein) to which an IgG antibody binds.

As used herein, the terms “IgG bispecific antibody”, “IgG BsAb”, “fully IgG bispecific antibody” or “fully IgG BsAb” refer to an antibody of the typical IgG architecture comprising two distinct Fabs, each of which direct binding to a separate antigen or antigenic determinant (i.e., different target proteins or different epitopes on the same target protein), and composed of two distinct IgG heavy chains and two distinct light chains. The V_{H}-C_{H}1 segment of one heavy chain associates with the V_{L}-C_{L} segment of one light chain to form a “first” Fab, wherein the V_{H} and V_{L} domains each comprise 3 CDRs which direct binding to a first antigen. The V_{H}-C_{H}1 segment of the second heavy chain associates with the V_{L}-C_{L} segment of the second light chain to form a “second” Fab, wherein the V_{H} and V_{L} domains each comprise 3 CDRs which direct binding to a second antigen that is different than the first. More particularly, the terms “IgG bispecific antibody”, “IgG BsAb”, “fully IgG bispecific antibody” or “fully IgG BsAb” refer to antibodies wherein the HC constant regions are composed of C_{H}1, C_{H}2, and C_{H}3 domains of the IgG1, IgG2 or IgG4 subtype, and particularly the human IgG1, human IgG2 or human IgG4. Even more particular, the terms refer to antibodies wherein the HC constant regions are composed of C_{H}1, C_{H}2, and C_{H}3 domains of the IgG1 or IgG4 subtype, and most particularly the human IgG1 or human IgG4 subtype. In addition, as used herein, the terms “IgG bispecific antibody”, “IgG BsAb”, “fully IgG bispecific antibody” and “fully IgG BsAb” refer to an antibody wherein the constant regions of each individual HC of the antibody are all of the same subtype (for example, each of the C_{H}1, C_{H}2, and C_{H}3 domains of a HC are all of the human IgG1 subtype, or each of the C_{H}1, C_{H}2, and C_{H}3 domains of a HC are all of the IgG2 subtype, or each of the C_{H}1, C_{H}2, and C_{H}3 domains of a HC are all of the IgG4 subtype.) Even more particular, the term refers to an antibody wherein the constant regions of both HCs are all of the same subtype (for example, both HCs have C_{H}1, C_{H}2, and C_{H}3 domains of the human IgG1 subtype, or both HCs have C_{H}1, C_{H}2, and C_{H}3 domains of the human IgG2 subtype, or both HCs have C_{H}1, C_{H}2, and C_{H}3 domains of the human IgG4 subtype).

As used herein, the terms “trispecific antibody”, “trispecific binding proteins”, “TsAb”, “IgG-Fab trispecific antibody”, “IgG-Fab TsAb”, “tandem Fab trispecific” or “tandem Fab TsAb” refer to an antibody comprising three distinct Fabs, each of which direct binding to a separate antigen or antigenic determinant (i.e., different target proteins or different epitopes on the same target protein). The three V_{H}-C_{H}1 segments of such TsAbs each associate with a V_{L}-C_{L} segment to form three Fabs, wherein the V_{H} and V_{L} domains each comprise 3 CDRs which direct binding to a first antigen. More particularly, the terms “trispecific antibody”, “trispecific binding proteins”, “TsAb”, “IgG-Fab trispecific antibody”, “IgG-Fab TsAb”, “tandem Fab trispecific” or “tandem Fab TsAb” refer to antibodies wherein the HC constant regions are composed of C_{H}1, C_{H}2, and/or CH3 domains of the IgG1, IgG2 or IgG4 subtype, and particularly the human IgG1, human IgG2 or human IgG4. Additionally, each C_{H}1 domain includes either the entire hinge or about the first five amino acids (e.g., EPKSC for IgG1) of the upper hinge to enable disulfide bonding with its cognate LC. Even more particular, the terms refer to antibodies wherein the HC constant regions are composed of C_{H}1, C_{H}2, and/or C_{H}3 domains of the IgG1 or IgG4 subtype, and most particularly the human IgG1 or human IgG4 subtype. In addition, as used herein, terms “trispecific antibody”, “TsAb”, “IgG-Fab trispecific antibody”, “IgG-Fab TsAb”, “tandem Fab trispecific” or “tandem Fab TsAb” refer to an antibody wherein all of the heavy chain constant regions of the antibody are all of the same subtype (for example, each of the C_{H}1, C_{H}2, and/or C_{H}3 domains within the TsAb are all of the human IgG1 subtype, or each of the C_{H}1, C_{H}2, and/or C_{H}3 domains are all of the IgG2 subtype, or each of the C_{H}1, C_{H}2, and/or C_{H}3 domains are all of the IgG4 subtype).

As used herein, the term “binding protein” or “trispecific binding protein” used in reference to a protein of the present invention refers to a TsAb as defined herein.

The processes and compounds of the present invention comprise designed amino acid modifications at particular residues within the constant and variable regions of heavy chain and light chain polypeptides. As one of ordinary skill in the art will appreciate, various numbering conventions may be employed for designating particular amino acid residues within IgG constant and variable region sequences. Commonly used numbering conventions include the “Kabat Numbering” and “EU Index Numbering” systems. “Kabat Numbering” or “Kabat Numbering system”, as used herein, refers to the numbering system devised and set forth by the authors in Kabat, et al., Sequences of Proteins of Immunological Interest, 5th Ed, Public Health Service, National Institutes of Health, Bethesda, MD (1991) for designating amino acid residues in both variable and constant domains of antibody heavy chains and light chains. “EU Index Numbering” or “EU Index Numbering system”, as used herein, refers to the numbering convention for designating amino acid residues in antibody heavy chain constant domains, and is also set forth in Kabat, et al., (1991). Other conventions that include corrections or alternate numbering systems for variable domains include Chothia (Chothia, C., Lesk, A. M. (1987), J Mol Biol 196: 901-917; Chothia, C., et al., (1989), Nature 342: 877-883), IMGT (Lefranc, et al., (2003), Dev Comp Immunol 27: 55-77), and Honegger (Honegger A., Pluckthun, A. (2001) J Mol Biol 309: 657-670). These references provide amino acid sequence numbering schemes for immunoglobulin variable regions that define the location of variable region amino acid residues of antibody sequences. Unless otherwise expressly stated herein, all references to immunoglobulin heavy chain variable region (i.e., V_{H}), constant region C_{H}1 and hinge amino acid residues (i.e., numbers) appearing herein are based on the Kabat Numbering system, as are all references to the light chain V_{L} and C_{L} residues. All references to immunoglobulin heavy chain constant region C_{H}2 and C_{H}3 residues (i.e., numbers) are based on the EU Index Numbering system. Thus, as used herein, the phrase “(according to Kabat Numbering)” indicates that the recited amino acid residue number (or position) is numbered in accordance with the Kabat Numbering system, whereas the phrase “(according to EU Index Numbering)” indicates that the recited amino acid residue number (or position) is numbered in accordance with the EU Index Numbering system. With knowledge of the residue number according to Kabat Numbering or EU Index Numbering, one of ordinary skill can apply the teachings of the art to identify amino acid sequence modifications within the present invention, according to any commonly used numbering convention. Note, while the present disclosure of the present invention employ Kabat Numbering or EU Index Numbering to identify particular amino acid residues, it is understood that the sequences appearing herein and/or accompanying the present application which are assigned a SEQ ID NO: and/or generated by Patent In Version 3.5 or higher, also may provide sequential numbering of amino acids within a given polypeptide and, thus, may not conform to the corresponding amino acid residue numbers as provided by Kabat Numbering or EU Index Numbering.

As used herein, the phrase “. . . a/an [amino acid name] substituted at residue . . .”, in reference to a heavy chain or light chain polypeptide, refers to substitution of the parental amino acid with the indicated amino acid. By way of example, a heavy chain comprising “a lysine substituted at residue 39” refers to a heavy chain wherein the parental amino acid sequence has been mutated to contain a lysine at residue number 39 in place of the parental amino acid. Such mutations may also be represented by denoting a particular amino acid residue number, preceded by the parental amino acid and followed by the replacement amino acid. For example, “Q39K” refers to a replacement of a glutamine at residue 39 with a lysine. Similarly, “39K” refers to replacement of a parental amino acid with a lysine. One of skill in the art will appreciate, however, that as a result of the interface design modifications of the present invention, Fab pairs and TsAbs (and processes for their preparation) are therefore provided wherein the component HC and LC amino acid sequences comprise the resulting or “replacement” amino acid at the designated residue. Thus, for example, a heavy chain which “comprises a lysine substituted at residue 39” may alternatively be denoted simply as a heavy chain which “comprises a lysine at residue 39.”

As used herein, the phrase “WT” or “WT sequence”, in reference to a HC or LC amino acid residue or polypeptide chain, refers to the wild-type or native amino acid or sequence of amino acids that naturally occupies the residue or residues of the polypeptide chain indicated.

An IgG-Fab TsAb, a tandem Fab TsAb, or Fab containing fragments thereof of the present invention can be produced using techniques well known in the art, such as recombinant expression in mammalian or yeast cells. In particular, the methods and procedures of the Examples herein may be readily employed. In addition, the IgG-Fab TsAbs, the tandem Fab TsAbs, or Fab containing fragments thereof of the present invention may be further engineered to comprise framework regions derived from fully human frameworks. A variety of different human framework sequences may be used in carrying out embodiments of the present invention. As a particular embodiment, the framework regions employed in the processes, as well as the IgG-Fab TsAbs, the tandem Fab TsAbs, or Fab containing fragments thereof of the present invention are of human origin or are substantially human (at least 95%, 97% or 99% of human origin.) The sequences of framework regions of human origin are known in the art and may be obtained from The Immunoglobulin Factsbook, by Marie-Paule Lefranc, Gerard Lefranc, Academic Press 2001, ISBN 012441351.

Expression vectors capable of directing expression of genes to which they are operably-linked are well known in the art. Expression vectors contain appropriate control sequences such as promoter sequences and replication initiation sites. They may also encode suitable selection markers as well as signal peptides that facilitate secretion of the desired polypeptide product(s) from a host cell. The signal peptide can be an immunoglobulin signal peptide or a heterologous signal peptide. Nucleic acids encoding desired polypeptides, for example the components of the IgG-Fab TsAbs, the tandem Fab TsAbs, or Fab containing fragments thereof prepared according to the processes of the present invention, may be expressed independently using the same or different promoters to which they are operably linked in a single vector or, alternatively, the nucleic acids encoding the desired products may be expressed independently using the same or different promoters to which they are operably linked in separate vectors. Single expression vectors encoding an IgG-Fab TsAb, a tandem Fab TsAb, or a Fab containing fragments thereof of the present invention may be prepared using standard methods.

As used herein, a “host cell” refers to a cell that is stably or transiently transfected, transformed, transduced or infected with nucleotide sequences encoding a desired polypeptide product or products. Creation and isolation of host cell lines producing an IgG-Fab TsAb, a tandem Fab TsAb, a Fab containing fragment thereof of the present invention of the present invention can be accomplished using standard techniques known in the art.

Mammalian cells are preferred host cells for expression of the IgG-Fab TsAbs, the tandem Fab TsAbs, or Fab containing fragments thereof according to the present invention. Particular mammalian cells include HEK293, NSO, DG-44, and CHO cells. Preferably, assembled proteins are secreted into the medium in which the host cells are cultured, from which the proteins can be recovered and isolated. Medium into which a protein has been secreted may be purified by conventional techniques. For example, the medium may be applied to and eluted from a Protein A or G column using conventional methods. Soluble aggregate and multimers may be effectively removed by common techniques, including size exclusion, hydrophobic interaction, ion exchange, hydroxyapatite or mixed modal chromatography. Recovered products may be immediately frozen, for example at −70° C., or may be lyophilized. As one of skill in the art will appreciate, when expressed in certain biological systems, e.g. mammalian cell lines, antibodies are glycosylated in the Fc region unless mutations are introduced in the Fc to reduce glycosylation. In addition, antibodies may be glycosylated at other positions as well.

The object of the present invention is to provide orthogonal interfaces which promote the correct pairing of particular heavy chain Fab fragments with their cognate light chain Fab fragments by introducing particular mutations into for example heavy chain C_{H}1/light chain C_{κ} domain pairs. As a result of the present invention, increased correct assembly of IgG TsAbs or Fab containing fragments is achieved, relative to the incorrectly assembled, when the individual heavy chain and light chain are concomitantly expressed in a host cell. The following Examples further illustrate the invention and provide typical methods and procedures for carrying out various particular embodiments of the present invention. However, it is understood that the Examples are set forth the by way of illustration and not limitation, and that various modifications may be made by one of ordinary skill in the art.

We describe the design, generation, and characterization of six TsAb formats with Fabs from pertuzumab, matuzumab, and MetMAb (i.e., HER-2×EGFR×cMet TsAbs) as well as the same six TsAb formats using Fabs from nivolumab, urelumab, and ipilimumab (i.e., PD-1×CD137×CTLA-4 TsAbs).

### EXAMPLES

### Example 1: Fab-based Trispecific Antibody Formats

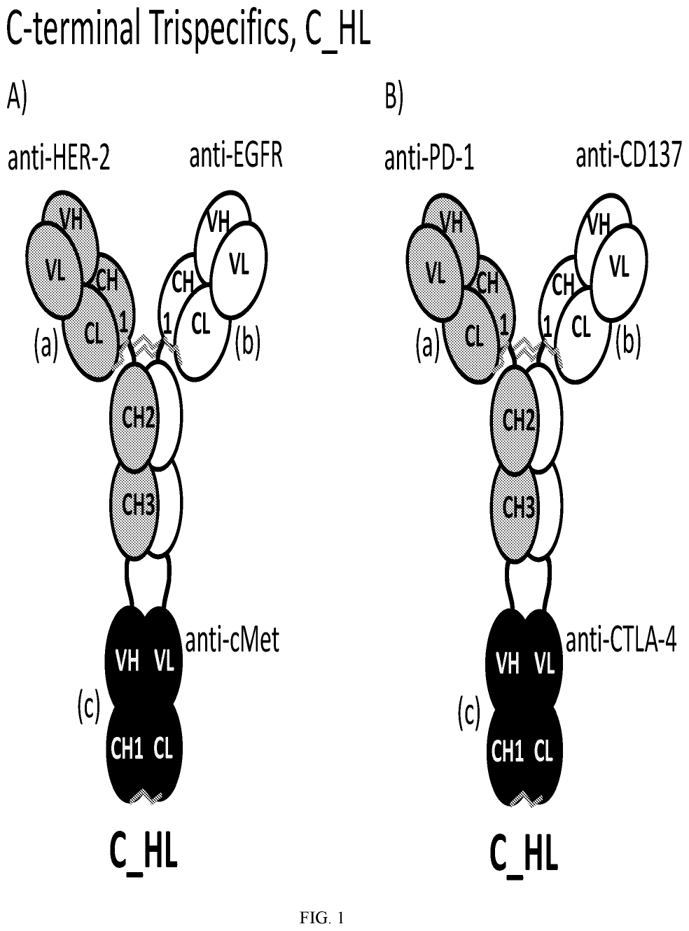

The Fab-based TsAbs were designed with varied geometries. Each construct contains three unique HC/LC moieties that must pair correctly to be fully functional. TsAbs were evaluated with or without an IgG Fc. The existence of the IgG-Fc moiety should impart improved pharmacokinetics through binding FcRn and recycling away from the lysosomal cellular compartment (Roopenian and Akilesh 2007). For Fc-containing TsAbs, we preferred to maintain a native-like antibody moiety within the TsAb construct and monovalent binding. We believe monovalent binding to each antigen would limit the overall molecular weight and ultimately allow for more precise affinity tuning to each target while reducing the potential for large immune complexes formation. To achieve monovalent binding of all three Fabs, we introduced an Fc heterodimerization design (20.8.34) from a previously described design effort (Leaver-Fay, et al., 2016). We evaluate both N- and C-terminal Fab fusions to the native-like antibody moiety. Schematic diagrams of the varied TsAb formats that were constructed are shown in FIG. 1-6. Some of the constructs covalently link HC and LC fragments in an effort to determine whether this might improve proper assembly. The IgG formats C-terminally fused with a Fab were denoted C_HL or C_L based on the polypeptide linkages that were evaluated, while the N-terminally fused TsAbs were denoted N_H and N_L. The non-IgG-Fc bearing TsAbs were made in a tandem Fab format and were denoted HHH and HHL. Drawings of and the amino acid sequences of the various HCs and LCs that comprise each of the TsAbs are provided herein.

To induce correct HC/LC pairing, multiple Fab interface designs were combined including previously described variable domain interface designs as well as redesigned CH1/Cλ and CH1/Cκ interfaces (Lewis, et al., 2014, Froning, et al., 2017). Without designs to induce specific HC/LC pairing, the expected percentage of correctly assembled TsAb for those without a heterodimeric Fc is <5%. The addition of a heterodimeric Fc to each TsAb reduces this expected assembly by greater than 50% given that ‘half-antibody’, non-covalently linked head-to-tail homodimer, can be a significant impurity on top of the normal homodimeric Fc species (Elliott, et al., 2014, Leaver-Fay, et al., 2016). However, all the Fc-containing TsAbs demonstrated between 10-50% half-antibody, which generally will need to be removed during manufacturing. N-terminally or C-terminally tethering Fabs to one of the HCs generally resulted in the HC without the tethered Fab expressing at a higher level, which led to half-antibody (data not shown). This half-antibody lacks the designed heterodimer interactions within the CH3 that stabilize the domain, which seemed to reveal itself with lower Tₘ peaks within the DSC curves of the Fc-containing TsAbs that contain between 10-50% half-antibody. Additional engineering or design work within the HC/LC and CH3 interfaces may be necessary within most of the constructs to reduce these impurities to make them acceptable from a manufacturing perspective. Additionally, one or more of the parental mAbs may be engineered to improve their expression and stability to equalize these attributes within the parental mAbs. Further reductions in the final expressed levels of mispaired and half-antibody products are possible even before purification. Also, other purification techniques may be applied such as hydrophobic interaction chromatography, ion exchange chromatography, or even mixed modal resins to reduce impurities. These have been used to eliminate mispairings and half-antibody for BsAbs. Ideally, such methods are individually tailored for each construct and applied once a particular format (FIG. 1-6) has been chosen for further characterization (Giese, et al., 2017).

Interestingly, there was an impact on antigen binding when Fabs within the TsAbs are recombinantly tethered at their VH or VL N-termini (Table 2(A) and (B)), similar to what was observed for the DVD-Ig format (Digiammarino, et al., 2011); however, only the C_HL format with dual tethering demonstrated kinetic on-rates impacted to the same extent as dually tethered DVD-Igs. The impact is most prevalent in the on-rate, although even off-rates were impacted for CTLA-4 binding even for TsAbs with single N-terminal tethers. These effects appear to be antigen-specific as only the dually tethered TsAb had a decrease in kinetic on-rate for cMet binding within the HER-2×EGFR×cMet TsAbs and EGFR and CD137 binding remained unperturbed across all the TsAbs even with some of these Fabs having an N-terminal tether. DVD-Ig on-rates can be improved by modifying the linker composition (Digiammarino, et al., 2011). Modification of linker composition for the TsAbs described here seems like a good next design step to improve the kinetic on-rates for each of the binding moieties.

Thus, achieving high levels of appropriately assembled Fab-based TsAbs using the structural designs disclosed here provides an important alternative to known interface designs and methods.

### Example 2: Subcloning and Transient CHO Expression

The amino acid sequences of the anti-HER-2 (pertuzumab), anti-EGFR (matuzumab), and anti-cMet (MetMAb) antibodies are known in the art (see, e.g., Lewis, et al., 2014). The anti-PD-1 (nivolumab), anti-CD137 (urelumab), and anti-CTLA-4 (ipilimumab) sequences are also known in the art (see, e.g., WO 2016/029073; US 2002/0086014). The Fab and full-length heavy chain (HC) coding regions were synthesized using PCR from previous constructs or large gblocks (IDT) with overlapping overhangs and polymerase chain reactions as described (Casimiro, et al., 1997, Lewis, et al., 2014). The Fab and HC pieces were assembled using a three-way ligation with the existing HindIII (5′) and EcoRl (3′) expression cassette restriction sites and an internal BamHI site designed within the (G₄S)₄ linker regions in a mammalian expression vector

(Lonza). The cognate Fab HCs and Fab light chains (LCs) were also constructed by overlapping gblock PCR and cloned into the same mammalian expression plasmid using the same restriction sites. The plasmid constructs for each TsAb were transiently transfected in 100 mL CHO for recombinant protein secretion into the media as described (Rajendra, et al., 2017). For all the TsAb formats, whether they required 5 plasmid transfection or 4 plasmid transfection, equal amounts of DNA were transfected for each plasmid up to a final DNA concentration of 3.2 μg/mL CHO cells (Rajendra, et al., 2017). Transfected cells were grown at 37 ° C. in a 5% CO₂ incubator while shaking at 125 r.p.m. for 5 days. Supernatants were harvested by centrifugation at 10 K r.p.m. for 5 minutes followed by passage through 2 μm filters.

Twelve TsAbs were produced at small scale to evaluate their expression and inherent assembly. Each of the TsAbs contained between four and five separate polypeptide chains that had to assemble properly to be fully functional and stable. All the TsAbs were transiently expressed at the 100 mL scale in CHO by co-transfection of single vectors encoding each of the particular HC and LC polypeptides and purified as described elsewhere herein and/or as is known in the art.

### Example 3: Purification and Biochemical Characterization

CHO supernatants containing each of the IgG-Fc containing TsAbs were loaded to a 5 ml HiTrap Mab select sure (GE) column at 5 mL/min using an AKTA explorer (GE Healthcare). The columns were washed using 25 mL phosphate buffered saline at pH 7.4 (PBS), followed by elution with 25 mL of a 20 mM citric acid buffer at pH 3.0 (all at 5 mL/min). CHO Supernatants containing TsAbs lacking an IgG-Fc were passed over a 5 mL Hitrap His (GE) at 5 mL/min. The column was washed with 25 mL PBS, followed by 25 ml of 25 mM imidazole in PBS, pH 7.5. The protein was eluted with 25 mL 0.5M imidazole in PBS. Each TsAb was further purified by passage over a Superdex 200 16 mm×60 cm column (Millipore Sigma) with PBS as the running buffer. The main peak based on UV_{280 nm} was collected and concentrated in PBS. The TsAbs were characterized biochemically by running 1-5 μg protein on SDS-PAGE gels (4-12% Bis-Tris) with the Mark 12 protein molecular weight standard (Invitrogen). Reducing conditions were induced by the addition of 1 μL 1 M DTT. Disulfide rearrangements under non-reducing conditions were blocked using N-ethylmaleimide (Taylor, et al., 2006). The oligomeric states of the TsAbs were assessed using analytical SEC. For each sample, between 20 to 60 μg material was injected over a Yarra-SEC-3000 analytical SEC HPLC (7.8×300 mm) column (Phenomenex) equilibrated in 10 mM phosphate, 150 mM NaCl, 0.02% NaN₃, pH 6.8, using an Agilent 1100 HPLC system equipped with an autosampler and in-line UV spectrometer. UV280 nm data was collected and analyzed using HPCHEM (Agilent).

The TsAbs all adopted their intended forms. Analysis on denaturing gels showed that each TsAb was predominately at the expected molecular weight. Under reducing conditions, the TsAbs all displayed the expected molecular weights for each of their polypeptide components (data not shown). The hydrodynamic behavior of the TsAbs was assessed using analytical SEC. The minor presence of ‘half-antibody’ (non-covalently linked Fc-homodimer (Elliott, et al., 2014, Leaver-Fay, et al., 2016)) can be observed in most of the Fc-containing TsAbs. The PD-1×CD137>CTLA-4 C_HL, C_L, and HHL proteins showed less ideal hydrodynamic behavior with multiple hydrodynamic species by SEC (data not shown). However, the remainder of the TsAbs, including all the HER-2×EGFR×cMet TsAbs, were predominately monodisperse with elution times within the expected range for their respective molecular weights (data not shown).

### Example 4: Mass Spectrometric Analysis of the Trispecific Antibodies

Intact mass spectrometry may be performed on the TsAbs to assess the level of correct chain pairing that should translate into their desired binding capacity. Briefly described, intact mass was measured using an Agilent 1290 HPLC coupled to an Agilent 6230 ESI-TOF mass spectrometer. Each sample (2 μg) was desalted on a MassPREP MicroDesalting Column 2.1×5 mm (Waters) loaded with 80% mobile phase A (0.2% Formic acid), and gradient eluted to 80% mobile phase B (0.2% Formic acid in acetonitrile) in 2 minutes and held for 1 minute. The same gradient was repeated as a wash step. The flow was bypassed for 3 minutes before the eluent was diverted into the mass spectrometer. The Agilent 6230 was run in positive ion mode at 4000 V, skimmer at 65 V, fragment or at 300 V, gas temperature at 350 ° C., dry gas at 12 psi and nebulizer gas at 40 psi. The MS scan was from 600 m/z to 4000 m/z with 1 scan/second. Data was collected from 3 minutes to 10 minutes and the protein molecular weight was determined by summing the TIC peak spectra followed by deconvolution with Agilent Bioconfirm Mass Hunter v7.0. The deconvolution for the non-reduced sample was from 80 to 180 kDa with a peak width of 1.5 Da using 20 iterations and a 1 Da step.

Mass spectrometry performed essentially as described above in this Example 4 (see, Table 1(A) and (B)). Main peaks at the appropriate molecular weights were clearly the predominate species in all the spectra (data not shown). The presence of half-antibody is apparent at different levels in all the Fc-containing TsAbs, but is absent, as expected, in the HHH and HHL formats (data not shown). The main peak was the expected species for every TsAb that was generated (data not shown), indicating that the HC/LC and Fc-heterodimer designs were able to influence proper HC/LC pairing far beyond what would be expected without any interference (−2-5%). With the understanding that different protein species may have different ionization potentials, the relative deconvoluted peak areas were still used to provide a semi-quantitative evaluation of the different species within the LCMS spectra. Using this method, none of the TsAbs fell below 50% correct HC/LC and HC/HC pairing (Table 1(A) and (B)). The majority of the TsAbs demonstrated >75% correct HC/LC and HC/HC assembly with a few achieving >90% correct assembly (Table 1(A) and (B)).

TABLE 1(A)
Intact mass analyses of the TsAbs

| | | | | | | | | |
|---|---|---|---|---|---|---|---|---|
| | | | Correct | | | | | |
| | Predicted | Observed | HC/LC | | Mispair | | Mispair2 | Half- |
| TsAb | MW | MW | pairing | Mispair1 | HC/LC | Mispair2 | HC/LC | Antibody |
| Protein | (kDa) | (kDa) | %ª | ID | %^{a} | ID | %ª | %^{a, b} |

HER-2 X EGFR X cMet TsAbs

| | | | | | | | | |
|---|---|---|---|---|---|---|---|---|
| TsAb_C_HL | 195526 | 195538 | 92.6 | 2xLC1 | 7.4 | n.d.^{c} | n.d. | 25 |
| TsAb_C_L | 194154 | 194160 | 100 | n.d. | n.d. | n.d. | n.d. | 27 |
| TsAb_N_H | 194359 | 194365 | 84.2 | 2xLC1 | 9.4 | 2xLC3 | 6.4 | 37 |
| | | | | 1xLC3 | | 1xLC2 | | |
| TsAb_N_L | 194359 | 194368 | 81 | 2xLC3 | 19 | n.d. | n.d. | 27 |
| | | | | 1xLC2 | | | | |
| TsAb_HHH | 145215 | 145218 | 77.8 | 2xLC2 | 8.3 | 2xLC1 | 13.9 | n.d. |
| | | | | 1xLC1 | | 1xLC3 | | |
| TsAb_HHL | 145215 | 145223 | 53.2 | 2xLC1 | 46.8 | n.d. | n.d. | n.d. |
| | | | | 1xLC3 | | | | |

^{a}% was calculated by the intensity of each peak assuming similar ionization propensity.
^{b}Half-antibody % was determined based on the non-deconvoluted intensity of the half-antibody divided by the non-deconvoluted intensity of the full-length TsAb.
^{c}n.d. = none detected. For TsAb_HHH and TsAb_HHL, half-antibody is not a possible by-product.

TABLE 1(B)
Intact mass analyses of the TsAbs

| | | | | | | | | |
|---|---|---|---|---|---|---|---|---|
| | | | Correct | | | | | |
| | Predicted | Observed | HC/LC | | Mispair | | Mispair2 | Half- |
| TsAb | MW | MW | pairing | Mispair1 | HC/LC | Mispair2 | HC/LC | Ab |
| Protein | (kDa) | (kDa) | %^{a} | ID | %^{a} | ID | %ª | %^{a, b} |

PD-1 X CD137 X CTLA-4 TsAbs

| | | | | | | | | |
|---|---|---|---|---|---|---|---|---|
| TsAb_C_HL | 193980 | 193982 | 93 | 2xLC2 | 7 | n.d. | n.d. | 11 |
| TsAb_C_L | 192592 | 192602 | 77 | 2xLC3 | 23 | n.d. | n.d. | 18 |
| | | | | 1xLC2 | | | | |
| TsAb_N_H | 192813 | 192876^{d} | 74 | Unclear | 26 | n.d. | n.d. | 50 |
| TsAb_N_L | 192796 | 192813 | 50.6 | 2xLC3 | 29.4 | 2xLC1 | 20 | 27 |
| | | | | 1xLC1 | | 1xLC3 | | |
| TsAb_HHH | 143669 | 143673 | 100 | n.d. | n.d. | n.d. | n.d. | n.d. |
| TsAb_HHL | 143652 | 143662 | 69 | 2xLC1 | 31 | n.d. | n.d. | n.d. |
| | | | | 1xLC3 | | | | |

ª% was calculated by the intensity of each peak assuming similar ionization propensity.
^{b}Half-antibody % was determined based on the non-deconvoluted intensity of the half-antibody divided by the non-deconvoluted intensity of the full-length TsAb.
^{c}n.d. = none detected. For TsAb_HHH and TsAb_HHL, half-antibody is not a possible by-product.

### Example 5: Binding Properties of the Trispecific Antibodies

Surface plasmon resonance (SPR) experiments may be performed to determine the binding properties of the trispecific binding proteins disclosed herein. Briefly stated, SPR may be performed on a Biacore3000 or Biacore8K (GE Healthcare) or similar device using an HBS-EP running buffer at 25 ° C. An anti-human kappa polyclonal antibody (Southern Biotech, cat. #2060-01) may be immobilized to 10,000 RU on CMS sensorchip surfaces using the standard amine coupling algorithms within the software. A reference surface may be prepared by direct immobilization using ethanolamine. Each TsAb for which binding properties are to be determined may be captured (100 nM) onto the chip surface by injection of 20 μL at a 5 μL/min flowrate. The antigens may be subsequently injected (50 nM in HBS-EP, 40 μL injections, 10 μL/min) at 10 minute intervals. Soluble human HER-2 and cMet extracellular domain (ECD) proteins may be purchased from Speed BioSystems (cat #: YCP1045 and YCP2247; His-tagged) and soluble human CTLA-4 ECD protein may be purchased from SinoBiologics (cat. #11159-H08H-50, His-tagged). Soluble human EGFR, PD-1, and CD137 ECD proteins (His-tagged) were produced in-house (Lewis, et al., 2014). The sensorchip surface may be regenerated by increasing the flowrate to 50 μL/min and performing 2 successive 5 μL injections of 0.1 M glycine, pH 1.5. Kinetic binding curves may be processed using the BiaEval software. Each curve may be double referenced using the matching ethanolamine surface curve and a separate kinetic curve may be performed entirely with buffer.

The TsAbs shown in FIGS. 1-6 were tested for their ability to bind their three respective antigens using SPR essentially as described above in this Example 5. Binding of all three antigens was apparent for all the TsAbs except the C_HL variants (Table 2(A) and (B)). Somehow, dually tethering the anti-cMet or anti-CTLA-4 Fabs at the C-terminus of the TsAb hinders the on-rate of the antigen significantly so that very little binding is observed (Table 2(A) and (B)). Releasing the dual tethering within the C_L format improves binding to the C-terminal Fab moiety; however, the fastest on-rates for cMet and CTLA-4 are to the TsAbs with N-terminal Fabs (N_ H and N_L), which are the only formats without a polypeptide linkage to the N-terminus of the VH or VL domains of these Fabs. While the level of antigen bound to each of the TsAbs appears to differ based on the format, the apparent dissociation kinetics for each antigen appears similar across all formats, except for the anti-CTLA-4 binding with Fabs that had an N-terminal tether. Somehow, adding an N-terminal tether impacts both association and dissociation of CTLA-4 to the ipilimumab Fab and this appears to be an antigen/epitope-specific phenomenon. Lastly, varying the order of antigen addition did not impact the kinetics of antigen binding for any of the TsAbs (data not shown). Overall, every TsAb format demonstrated strong trispecific binding except the C_HL format whose kinetic on-rate was hindered by dual tethering.

TABLE 2
Kinetic analyses of the TsAbs and their parental mAbs using surface plasmon resonance
(A)
HER-2 × EGFR × cMet TsAbs

| | HER-2ª | HER-2ª | HER-2ª | EGFRª | EGFRª | EGFRª | cMET | cMet | cMet |
|---|---|---|---|---|---|---|---|---|---|
| | kₐ | k_{d} | K_{D} | kₐ | k_{d} | K_{D} | kₐ | k_{d} | K_{D} |
| TsAb | (s⁻¹M⁻¹) | (s⁻¹) | (M) | (s⁻¹M⁻¹) | (s⁻¹) | (M) | (s⁻¹M⁻¹) | (s⁻¹) | (M) |
| Pertuzumab | 2.1e5 | 1.1e−4 | 0.5e−9 | | | | | | |
| Matuzumab | | | | 6.5e5 | 8.3e−3 | 1.3e−8 | | | |
| MetMAb | | | | | | | 7.5e4 | 1.0e−4 | 1.3e−9 |
| TsAb_C_HL | 2.3e5 | 2.7e−4 | 1.2e−9 | 8.5e5 | 8.5e−3 | 1.0e−8 | 3.4e3 | 2.5e−4 | 7.4e−8 |
| TsAb_C_L | 5.2e5 | 3.9e−4 | 0.8e−9 | 1.9e6 | 8.6e−3 | 4.5e−9 | 3.6e5 | 2.5e−4 | 6.9e−10 |
| TsAb_N_H | 2.3e5 | 2.6e−4 | 1.1e−9 | 6.8e5 | 8.5e−3 | 1.3e−8 | 5.2e5 | 1.6e−4 | 3.1e−10 |
| TsAb_N_L | 2.6e5 | 2.4e−4 | 0.9e−9 | 7.2e5 | 9.0e−3 | 1.3e−8 | 4.4e5 | 1.6e−4 | 3.6e−10 |
| TsAb_HHH | 2.6e5 | 2.2e−4 | 0.8e−9 | 3.6e5 | 5.5e−3 | 1.5e−8 | 1.7e5 | 1.3e−4 | 7.7e−10 |
| TsAb_HHL | 3.0e5 | 2.1e−4 | 0.7e−9 | 5.6e5 | 5.8e−3 | 1.0e−8 | 2.6e5 | 1.4e−4 | 5.4e−10 |

(B)
PD-1 × CD137 × CTLA-4 TsAbs

| | PD-1 | PD-1 | PD-1 | CD137 | CD137 | CD137 | CTLA-4 | CTLA-4 | CTLA-4 |
|---|---|---|---|---|---|---|---|---|---|
| | kₐ | k_{d} | K_{D} | kₐ | k_{d} | K_{D} | kₐ | k_{d} | K_{D} |
| TsAb | (s⁻¹M⁻¹) | (s⁻¹) | (M) | (s⁻¹M⁻¹) | (s⁻¹) | (M) | (s⁻¹M⁻¹) | (s⁻¹) | (M) |
| Nivolumab | 9.2e4 | 1.3e−3 | 1.4e−8 | | | | | | |
| Urelumab | | | | 2.1e5 | 2.3e−3 | 1.1e−8 | | | |
| Ipilimumab | | | | | | | 3.2e5 | 0.9e−4 | 2.7e−10 |
| TsAb_C_HL | 3.0e5 | 2.9e−3 | 9.7e−9 | 5.3e5 | 2.5e−3 | 4.7e−9 | 4.1e4 | 1.0e−3 | 2.4e−8 |
| TsAb_C_L | 4.1e5 | 2.5e−3 | 6.1e−9 | 6.2e5 | 2.4e−3 | 3.9e−9 | 7.3e4 | 3.1e−4 | 4.2e−9 |
| TsAb_N_H | 2.0e5 | 2.7e−3 | 1.4e−8 | 4.4e5 | 2.4e−3 | 5.5e−9 | 4.4e5 | 1.4e−4 | 3.2e−10 |
| TsAb_N_L | 2.3e5 | 2.8e−3 | 1.2e−8 | 4.4e5 | 2.2e−3 | 5.0e−9 | 3.7e5 | 1.5e−4 | 4.1e−10 |
| TsAb_HHH | 1.2e5 | 2.4e−3 | 2.0e−8 | 2.8e5 | 1.7e−3 | 6.1e−9 | 2.7e5 | 1.2e−3 | 4.4e−9 |
| TsAb_HHL | 2.3e5 | 2.3e−3 | 1.0e−8 | 3.0e5 | 1.9e−3 | 6.3e−9 | 7.5e4 | 1.4e−3 | 1.9e−8 |

ªValues for pertuzumab were reported previously (Lewis, et al., 2014).

Taken together, the methods described here provide alternative approaches to generating binding proteins with significantly improved cell-specific binding, receptor cross-linking, blockade of tumor escape mechanisms, and/or immune cell redirection. Furthermore, the TsAbs of the present invention could have a positive impact in many disease indications that are in need of improved therapies. The TsAbs of the present invention use native-like antibody Fab domains, as opposed to antibody Fv, domain antibody, or alternative scaffold proteins, to achieve the desired multivalent binding. Given the very low theoretical assembly levels expected in the absence of interface designs (<2% for the IgG Fc-containing TsAbs), the level of correct assembly achieved within these initial proof-of-concept TsAbs is extraordinarily high. Generally, >80% correct assembly would allow the TsAbs to be produced without complex post-expression purification schemes. Promisingly, half of the TsAbs demonstrated >80% correct HC/LC and HC/HC assembly with a few >90%. All of the Fc-containing TsAbs demonstrated between 10-50% half-antibody, which ideally would be removed during manufacturing for clinical use. N-terminally or C-terminally tethering Fabs to one of the HCs generally resulted in the HC without the tethered Fab expressing at a higher level, which led to half-antibody (data not shown). There can also be an impact on antigen binding when Fabs within the TsAbs are recombinantly tethered at their VH or VL N-termini (Table 2(A) and (B)). This is similar to what was observed for the DVD-Ig format (Digiammarino, et al., 2011); however, only the C_HL format with dual tethering described herein demonstrated kinetic on-rates impacted to the same extent as dually tethered DVD-Igs. The impact is most prevalent in the on-rate, although even off-rates were impacted for CTLA-4 binding even for TsAbs with single N-terminal tethers. These effects appear to be antigen-specific as only the dually tethered TsAb had a decrease in kinetic on-rate for cMet binding within the HER-2×EGFR×cMet TsAbs and EGFR and CD137 binding remained unperturbed across all the TsAbs even with some of these Fabs having an N-terminal tether.

### LISTING OF AMINO ACID AND NUCLEOTIDE SEQUENCES

LC/HC and C_{H}3 heterodimer designed amino acid substitutions are underlined and in bold, respectively. Complementarity determining region (CDR) sequences are shaded in gray.

Sequences for FIG. 1(A): HER-2 X EGFR X cMet TsAb_C_HL

SEQ ID NO: 1 (HC1)

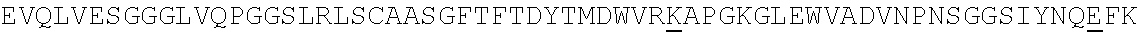

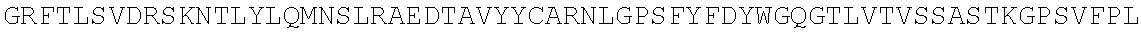

APSSKSTSGGTAALGCLVKDYFPEPVTVSWNSGALTSGVATGPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKENWYVDGVEVHNAKTKPREQYQSTYRVVSVLTVLHQDWLNGKEYKCKVS
NKALPAPIEKTISKAKGQPREPQVSTLPPSREEMTKNQVSLMCLVYGFYPSDIAVEWESNGQPEN
NYKTTPPVLDSDGSFFLYSVLTVDKSRWQQGNVFSCSVMHEALHNHYTQKSLSLSPGKGGGGSGG

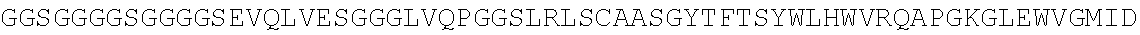

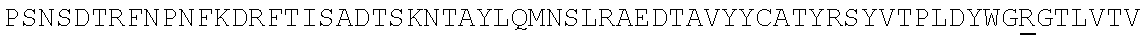

SSASTKGPSVFPLAPSSKSTSGGTAALGCLVADYFPEPVTVSWNSGALTSGVHTFPAVLQSSGLY
SLSSVVTVPSSSLGTQTYICNVNHKPSNTKVDERVEPKSC
SEQ ID NO: 2 (LC1)(HER-2)

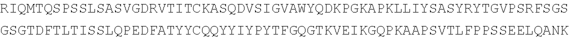

ATLVCYISDEYPGAVTVAWKADSSPVKAGVETTTPSKQSNNKYAAWSYLSLTPEQWKSHRSYSCQ
VTHEGSTVEKTVAPTEC
SEQ ID NO: 3 (HC2)

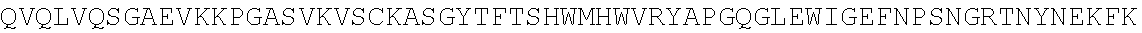

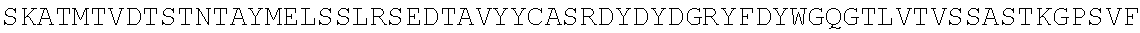

PLAPCSKSTSGGTAALGCLVKDYFPEPVTVSWNSGALTSGVHTFPAVLQSSGLYSLSSVVTVPSS
SLGTQTYICNVNHKPSNTKVDKKVEPDSGDKTHTCPPCPAPEAAGGPSVFLFPPKPKDTLMISRT
PEVTCVVVDVSHEDPEVKENWYVDGVEVHNAKTKPREEQYQSTYRVVSVLTVLHQDWLNGKEYKC
KVSNKALPAPIEKTISKAKGQPREPQVYTLPPSRGDMTKNQVQLTCLVKGFYPSDIAVEWESNGQ
PENNYKTTPPVLDSDGSFFLASKLTVDKSRWQQGNVFSCSVMHEALHNHYTQKSLSLSPGKGGGG

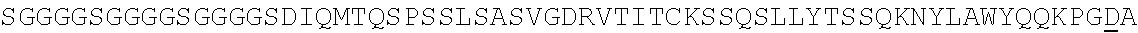

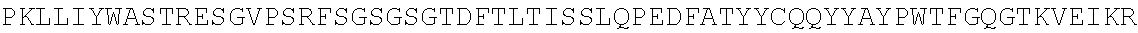

TVAAPSVFIFPPSDKQLKSGTARVVCLLNNFYPREAKVQWKVDNALQSGNSQESVTEQDSKDSTY
SLISTLTLSKADYEKHKVYACEVTHQGLSSPVTKSENRGEC
SEQ ID NO: 4 (LC2)(EGFR)

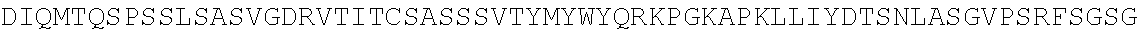

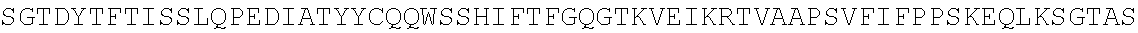

VVCLLNNFYPREAKVQWKVDNALQSGNSQESVTEQDSKDSTYSLSSTLTLSKADYEKHKVYACEV
THQGLSSPVTKSENRGEC
Sequences for FIG. 1(B): PD-1 X CD137 X CTLA-4 TsAb_C_HL
SEQ ID NO: 5 (HC1)

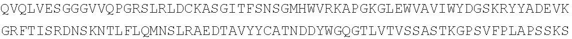

TSGGTAALGCLVKDYFPEPVTVSWNSGALTSGVATGPAVLQSSGLYSLSSVVTVPSSSLGTQTYI
CNVNHKPSNTKVDKKVEPKSCDKTHTCPPCPAPEAAGGPSVFLFPPKPKDTLMISRTPEVTCVVV
DVSHEDPEVKENWYVDGVEVHNAKTKPREEQYQSTYRVVSVLTVLHQDWLNGKEYKCKVSNKALP
APIEKTISKAKGQPREPQVSTLPPSREEMTKNQVSLMCLVYGFYPSDIAVEWESNGQPENNYKTT
PPVLDSDGSFFLYSVLTVDKSRWQQGNVFSCSVMHEALHNHYTQKSLSLSPGKGGGGGGGGSGG

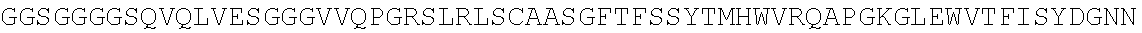

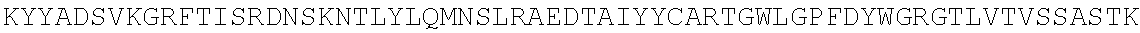

GPSVFPLAPSSKSTSGGTAALGCLVADYFPEPVTVSWNSGALTSGVHTFPAVLQSSGLYSLSSVV
TVPSSSLGTQTYICNVNHKPSNTKVDERVEPKSC
SEQ ID NO: 6 (LC1)(PD-1)

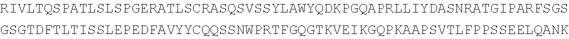

ATLVCYISDEYPGAVTVAWKADSSPVKAGVETTTPSKQSNNKYAAWSYLSLTPEQWKSHRSYSCQ
VTHEGSTVEKTVAPTEC
SEQ ID NO: 7 (HC2)

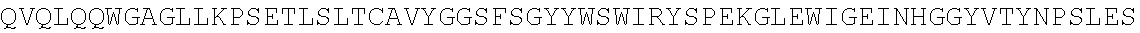

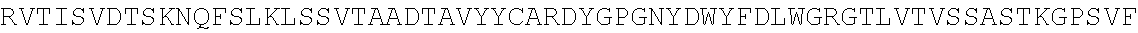

PLAPCSKSTSGGTAALGCLVKDYFPEPVTVSWNSGALTSGVHTFPAVLQSSGLYSLSSVVTVPSS
SLGTQTYICNVNHKPSNTKVDKKVEPDSGDKTHTCPPCPAPEAAGGPSVELEPPKPKDTLMISRT
PEVTCVVVDVSHEDPEVKENWYVDGVEVHNAKTKPREEQYQSTYRVVSVLTVLHQDWLNGKEYKC
KVSNKALPAPIEKTISKAKGQPREPQVYTLPPSRGDMTKNQVQLTCLVKGFYPSDIAVEWESNGQ
PENNYKTTPPVLDSDGSFFLASKLTVDKSRWQQGNVFSCSVMHEALHNHYTQKSLSLSPGKGGGG

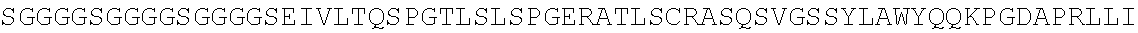

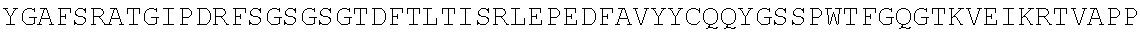

SVFIFPPSDKQLKSGTARVVCLLNNFYPREAKVQWKVDNALQSGNSQESVTEQDSKDSTYSLIST
LTLSKADYEKHKVYACEVTHQGLSSPVTKSENRGEC
SEQ ID NO: 8 (LC2)(CD137)

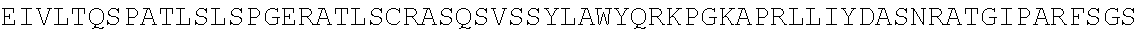

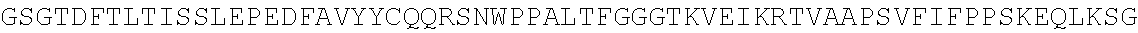

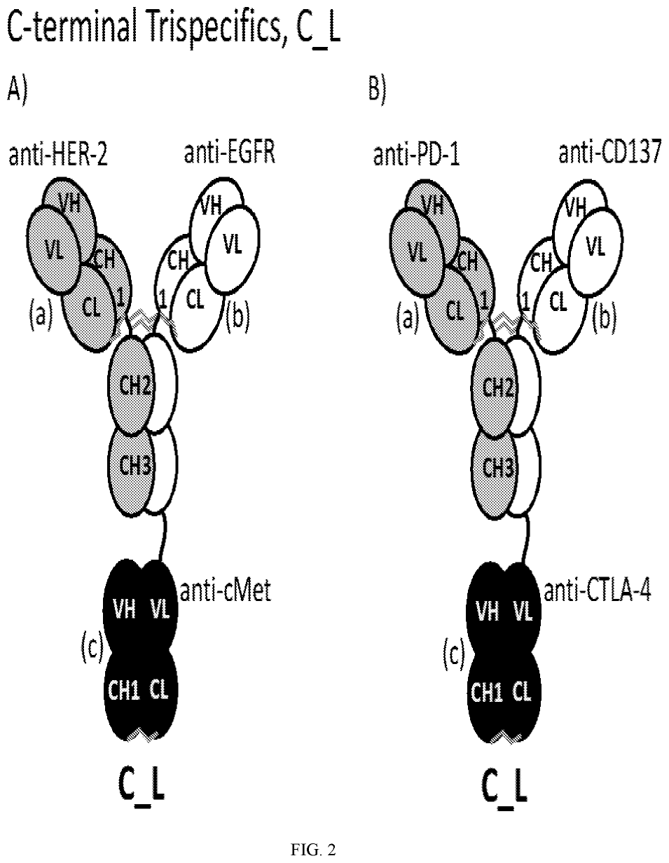

TASVVCLLNNFYPREAKVQWKVDNALQSGNSQESVTEQDSKDSTYSLSSTLTLSKADYEKHKVYA
CEVTHQGLSSPVTKSENRGEC
Sequences for FIG. 2(A): HER-2 X EGFR X cMet TsAb_C_L
SEQ ID NO: 9 (HC1)(HER-2)

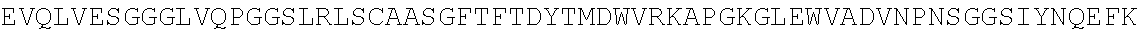

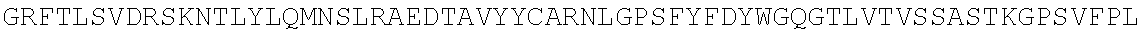

APSSKSTSGGTAALGCLVKDYFPEPVTVSWNSGALTSGVATGPAVLQSSGLYSLSSVVTVPSSSL
GTQTYICNVNHKPSNTKVDKKVEPKSCDKTHTCPPCPAPEAAGGPSVFLFPPKPKDTLMISRTPE
VTCVVVDVSHEDPEVKENWYVDGVEVHNAKTKPREEQYQSTYRVVSVLTVLHQDWLNGKEYKCKV
SNKALPAPIEKTISKAKGQPREPQVSTLPPSREEMTKNQVSLMCLVYGFYPSDIAVEWESNGQPE
NNYKTTPPVLDSDGSFFLYSVLTVDKSRWQQGNVFSCSVMHEALHNHYTQKSLSLSPG
SEQ ID NO: 2 (LC1)(HER-2)
SEQ ID NO: 10 (VH)(cMET)

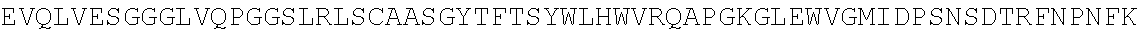

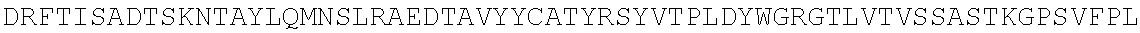

APSSKSTSGGTAALGCLVADYFPEPVTVSWNSGALTSGVHTFPAVLQSSGLYSLSSVVTVPSSSL
GTQTYICNVNHKPSNTKVDERVEPKSC
SEQ ID NO: 3 (HC2)
SEQ ID NO: 4 (LC2)(EGFR)
Sequences for FIG. 2(B): PD-1 X CD137 X CTLA-4 TsAb_C_L
SEQ ID NO: 11 (HC1)(PD-1)

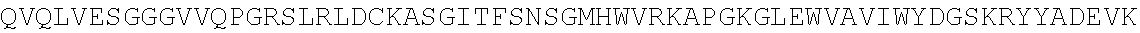

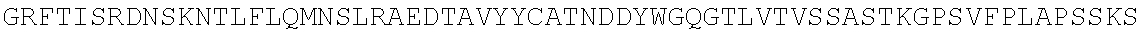

TSGGTAALGCLVKDYFPEPVTVSWNSGALTSGVATGPAVLOSSGLYSLSSVVTVPSSSLGTQTYI
CNVNHKPSNTKVDKKVEPKSCDKTHTCPPCPAPEAAGGPSVFLFPPKPKDTLMISRTPEVTCVVV
DVSHEDPEVKENWYVDGVEVHNAKTKPREEQYQSTYRVVSVLTVLHQDWLNGKEYKCKVSNKALP
APIEKTISKAKGQPREPQVSTLPPSREEMTKNQVSLMCLVYGFYPSDIAVEWESNGQPENNYKTT
PPVLDSDGSFFLYSVLTVDKSRWQQGNVESCSVMHEALHNHYTQKSLSLSPG
SEQ ID NO: 6 (LC1)(PD-1)
SEQ ID NO: 12 (VH)(CTLA-4)

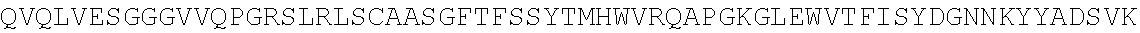

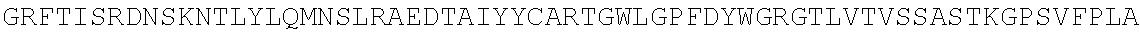

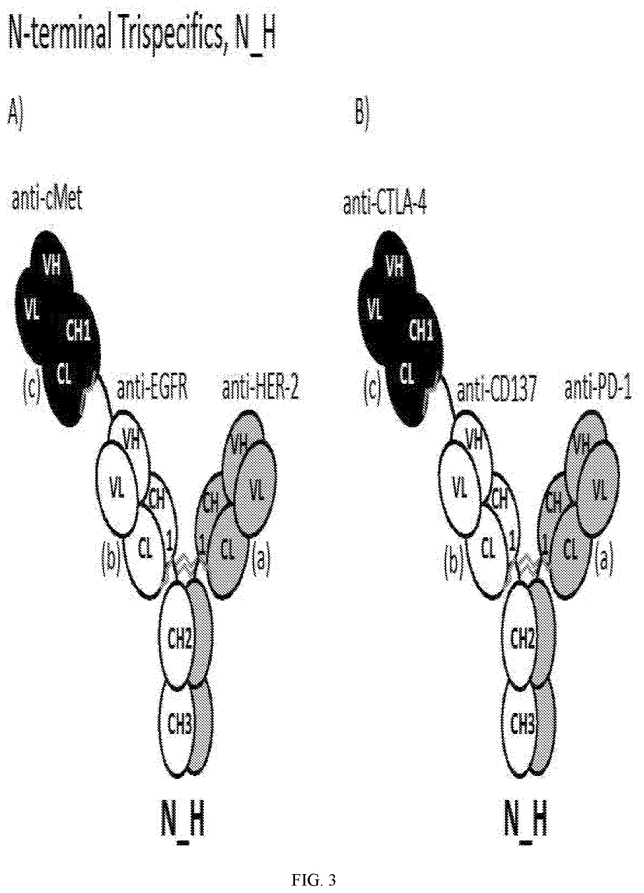

PSSKSTSGGTAALGCLVADYFPEPVTVSWNSGALTSGVHTEPAVLQSSGLYSLSSVVTVPSSSLG
TQTYICNVNHKPSNTKVDERVEPKSC
SEQ ID NO: 7 (HC2)
SEQ ID NO: 8 (LC2)(CD137)
Sequences for FIG. 3(A): HER-2 X EGFR X cMet TsAb_N_H
SEQ ID NO: 13 (HC1)

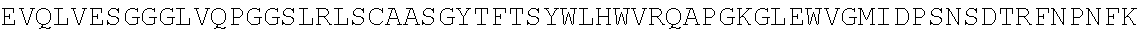

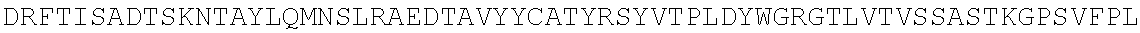

APSSKSTSGGTAALGCLVADYFPEPVTVSWNSGALTSGVHTFPAVLQSSGLYSLSSVVTVPSSSL
GTQTYICNVNHKPSNTKVDERVEPKSCGGGGSGGGGSGGGGSGGGGSGGGGSQVQLVQSGAEVKK

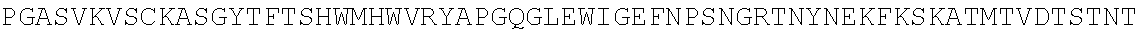

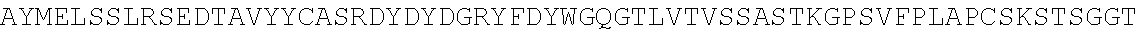

AALGCLVKDYFPEPVTVSWNSGALTSGVHTFPAVLQSSGLYSLSSVVTVPSSSLGTQTYICNVNH
KPSNTKVDKKVEPDSGDKTHTCPPCPAPEAAGGPSVFLFPPKPKDTLMISRTPEVTCVVVDVSHE
DPEVKENWYVDGVEVHNAKTKPREEQYQSTYRVVSVLTVLHQDWLNGKEYKCKVSNKALPAPIEK
TISKAKGQPREPQVYTLPPSRGDMTKNQVQLTCLVKGFYPSDIAVEWESNGQPENNYKTTPPVLD
SDGSFFLASKLTVDKSRWQQGNVFSCSVMHEALHNHYTQKSLSLSPG
SEQ ID NO: 4 (LC1)(EGFR)
SEQ ID NO: 9 (HC2)(HER-2)
SEQ ID NO: 2 (LC2)(HER-2)
SEQ ID NO: 14 (LC3)(cMET)

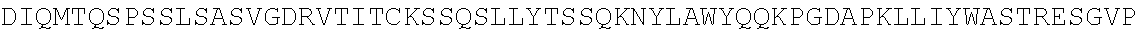

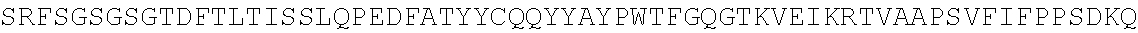

LKSGTARVVCLINNFYPREAKVQWKVDNALQSGNSQESVTEQDSKDSTYSLISTLTLSKADYEKH
KVYACEVTHQGLSSPVTKSENRGEC
Sequences for FIG. 3(B): PD-1 X CD137 X CTLA-4 TsAb_N_H
SEQ ID NO: 15 (HC1)

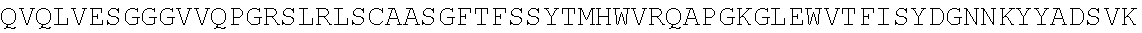

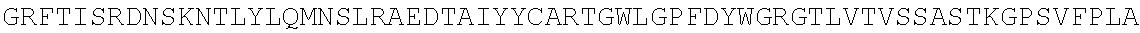

PSSKSTSGGTAALGCLVADYFPEPVTVSWNSGALTSGVHTFPAVLQSSGLYSLSSVVTVPSSSLG
TQTYICNVNHKPSNTKVDERVEPKSCGGGGSGGGGSGGGGSGGGGSGGGGSQVQLQQWGAGLLKP

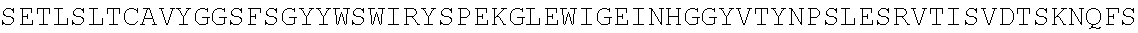

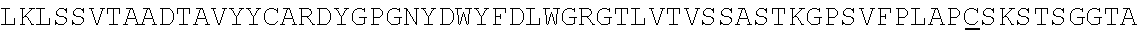

ALGCLVKDYFPEPVTVSWNSGALTSGVHTFPAVLQSSGLYSLSSVVTVPSSSLGTQTYICNVNHK
PSNTKVDKKVEPDSGDKTHTCPPCPAPEAAGGPSVFLFPPKPKDTLMISRTPEVTCVVVDVSHED
PEVKENWYVDGVEVHNAKTKPREEQYQSTYRVVSVLTVLHQDWLNGKEYKCKVSNKALPAPIEKT
ISKAKGQPREPQVYTLPPSRGDMTKNQVQLTCLVKGFYPSDIAVEWESNGQPENNYKTTPPVLDS
DGSFFLASKLTVDKSRWQQGNVESCSVMHEALHNHYTQKSLSLSPG
SEQ ID NO: 8 (LC1)(CD137)
SEQ ID NO: 11 (HC2)(PD-1)
SEQ ID NO: 6 (LC2)(PD-1)
SEQ ID NO: 16 (LC3)(CTLA-4)

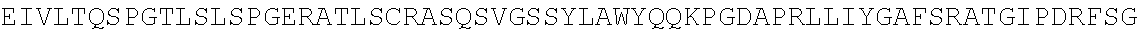

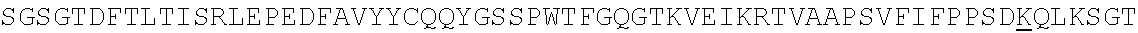

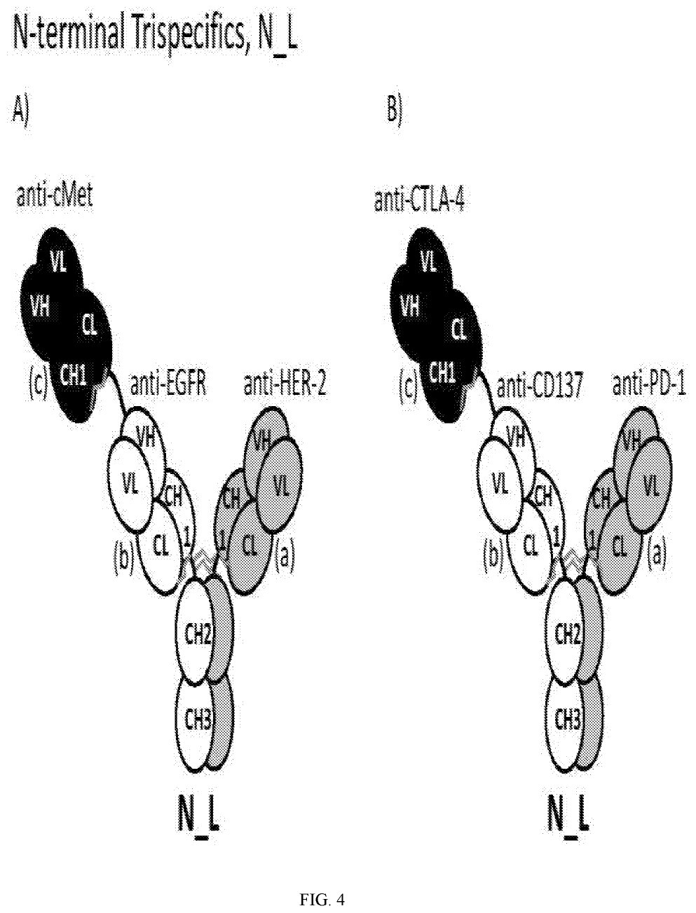

ARVVCLLNNFYPREAKVQWKVDNALQSGNSQESVTEQDSKDSTYSLISTLTLSKADYEKHKVYAC
EVTHQGLSSPVTKSENRGEC
Sequences for FIG. 4(A): HER-2 X EGFR X cMet TsAb_N_L
SEQ ID NO: 17 (HC1)

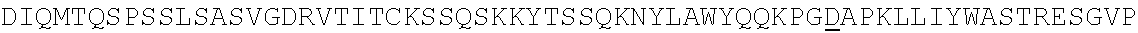

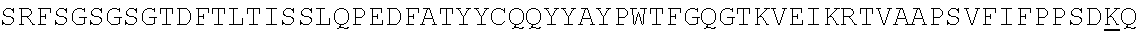

LKSGTARVVCLLNNFYPREAKVOWKVDNALQSGNSQESVTEQDSKDSTYSLISTLTLSKADYEKH
KVYACEVTHQGLSSPVTKSFNRGECGGGGSGGGGSGGGGSGGGGSGGGGSQVQLVQSGAEVKKPG

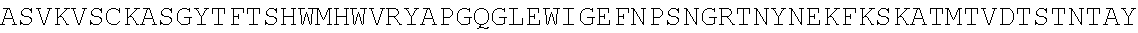

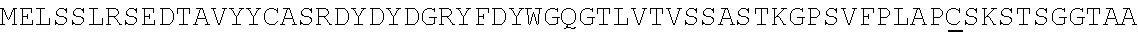

LGCLVKDYFPEPVTVSWNSGALTSGVHTFPAVLQSSGLYSLSSVVTVPSSSLGTQTYICNVNHKP
SNTKVDKKVEPDSGDKTHTCPPCPAPEAAGGPSVFLFPPKPKDTLMISRTPEVTCVVVDVSHEDP
EVKFNWYVDGVEVHNAKTKPREEQYQSTYRVVSVLTVLHQDWLNGKEYKCKVSNKALPAPIEKTI
SKAKGQPREPQVYTLPPSRGDMTKNQVQLTCLVKGFYPSDIAVEWESNGQPENNYKTTPPVLDSD
GSFFLASKLTVDKSRWQQGNVFSCSVMHEALHNHYTQKSLSLSPG
SEQ ID NO: 4 (LC1)(EGFR)
SEQ ID NO: 9 (HC2)(HER-2)
SEQ ID NO: 2 (LC2)(HER-2)
SEQ ID NO: 10 (VH)(cMET)
Sequences for FIG. 4(B): PD-1 X CD137 X CTLA-4 TsAb_N_L
SEQ ID NO: 18 (HC1)

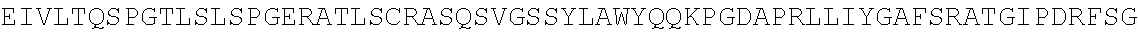

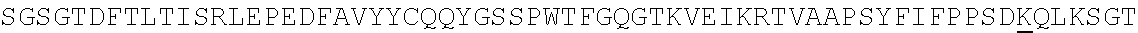

ARVVCLLNNEYPREAKVQWKVDNALQSGNSQESVTEQDSKDSTYSLISTLTLSKADYEKHKVYAC
EVTHQGLSSPVTKSENRGECGGGGSGGGGSGGGGSGGGGSGGGGSQVQLQQWGAGLLKPSETLSL

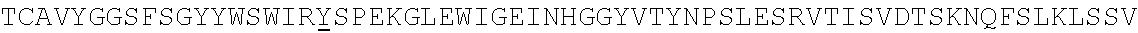

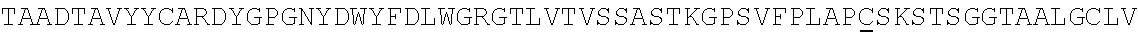

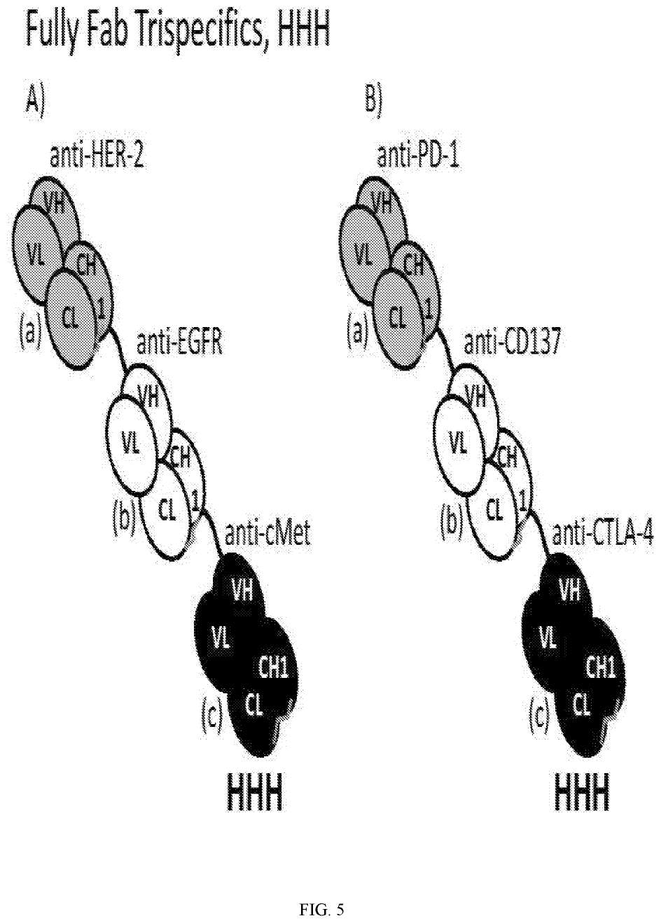

KDYFPEPVTVSWNSGALTSGVHTFPAVLQSSGLYSLSSVVTVPSSSLGTQTYICNVNHKPSNTKV
DKKVEPDSGDKTHTCPPCPAPEAAGGPSVFLFPPKPKDTLMISRTPEVTCVVVDVSHEDPEVKEN
WYVDGVEVHNAKTKPREEQYQSTYRVVSVLTVLHQDWLNGKEYKCKVSNKALPAPIEKTISKAKG
QPREPQVYTLPPSRGDMTKNQVQLTCLVKGFYPSDIAVEWESNGQPENNYKTTPPVLDSDGSFEL
ASKLTVDKSRWQQGNVFSCSVMHEALHNHYTQKSLSLSPG
SEQ ID NO: 8 (LC1)(CD137)
SEQ ID NO: 11 (HC2)(PD-1)
SEQ ID NO: 6 (LC2)(PD-1)
SEQ ID NO: 12 (VH)(CTLA-4)
Sequences for FIG. 5(A): HER-2 X EGFR X cMet TsAb_HHH
SEQ ID NO: 19 (HC1)

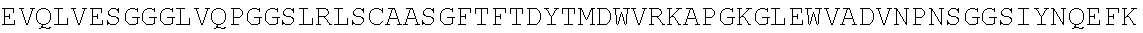

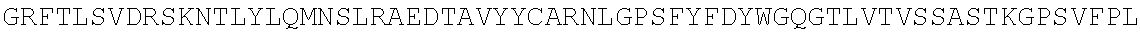

APSSKSTSGGTAALGCLVKDYFPEPVTVSWNSGALTSGVATGPAVLQSSGLYSLSSVVTVPSSSL

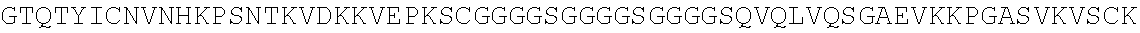

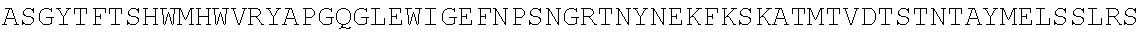

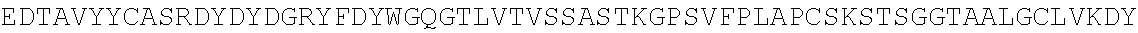

FPEPVTVSWNSGALTSGVHTFPAVLQSSGLYSLSSVVTVPSSSIGTQTYICNVNHKPSNTKVDKK

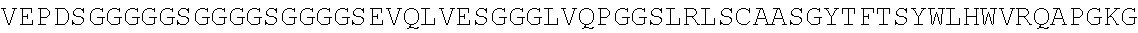

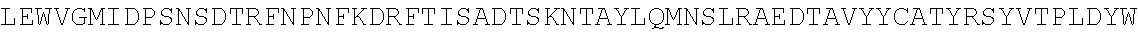

GRGTLVTVSSASTKGPSVFPLAPSSKSTSGGTAALGCLVADYFPEPVTVSWNSGALTSGVHTFPA
VLQSSGLYSLSSVVTVPSSSLGTQTYICNVNHKPSNTKVDERVEPKSCHHHHHHHH
SEQ ID NO: 2 (LC1)(HER-2)
SEQ ID NO: 4 (LC2)(EGFR)
SEQ ID NO: 14 (LC3)(cMET)
Sequences for FIG. 5(B): PD-1 X CD137 X CTLA-4 TsAb_HHH
SEQ ID NO: 20 (HC1)

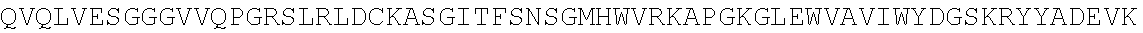

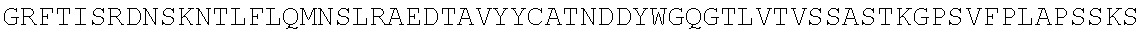

TSGGTAALGCLVKDYFPEPVTVSWNSGALTSGVATGPAVLQSSGLYSLSSVVTVPSSSLGTQTYI

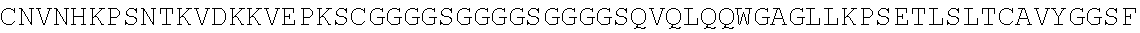

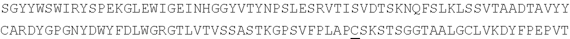

VSWNSGALTSGVHTFPAVLQSSGLYSLSSVVTVPSSSLGTQTYICNVNHKPSNTKVDKKVEPDSG

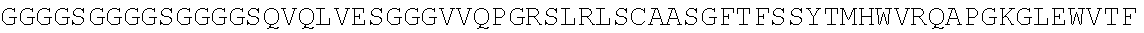

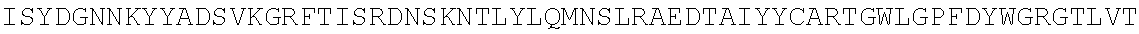

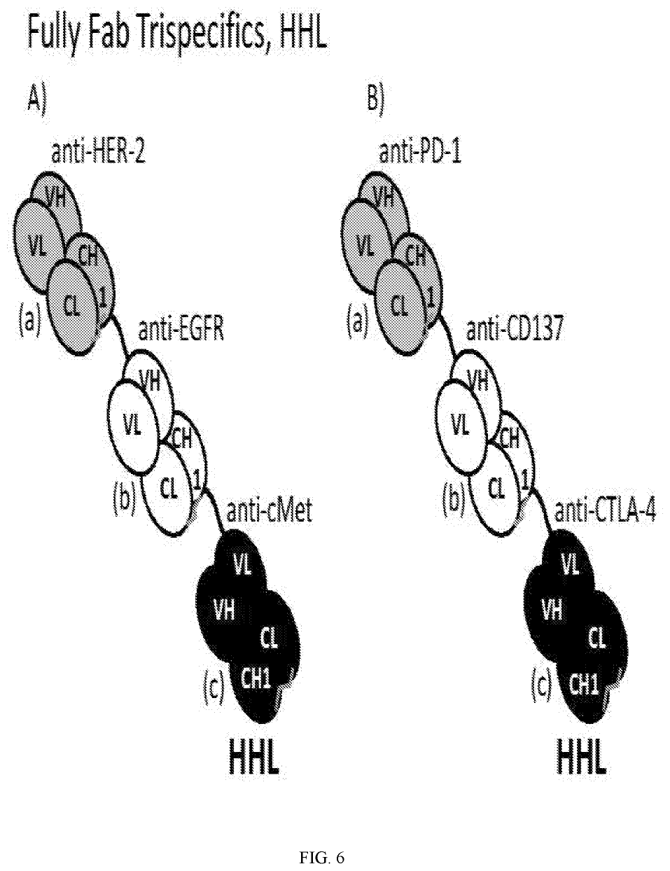

VSSASTKGPSVFPLAPSSKSTSGGTAALGCLVADYFPEPVTVSWNSGALTSGVHTFPAVLQSSGL
YSLSSVVTVPSSSLGTQTYICNVNHKPSNTKVDERVEPKSCHHHHHHHH
SEQ ID NO: 6 (LC1)(PD-1)
SEQ ID NO: 8 (LC2)(CD137)
SEQ ID NO: 16 (LC3)(CTLA-4)
Sequences for FIG. 6(A): HER-2 X EGFR X cMet TsAb_HHL
SEQ ID NO: 21 (HC1)

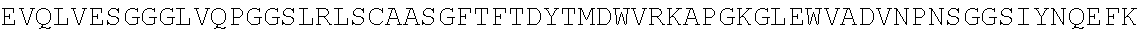

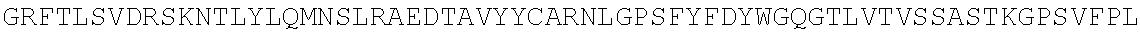

APSSKSTSGGTAALGCLVKDYFPEPVTVSWNSGALTSGVATGPAVLOSSGLYSLSSVVTVPSSSL

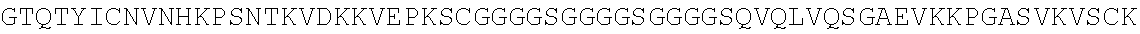

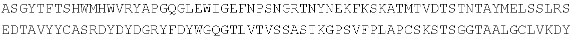

FPEPVTVSWNSGALTSGVHTFPAVLQSSGLYSLSSVVTVPSSSLGTQTYICNVNHKPSNTKVDKK

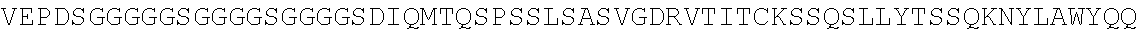

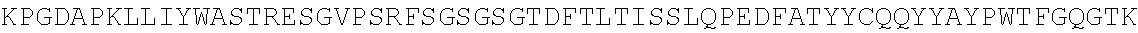

VEIKRTVAAPSVFIFPPSDKQLKSGTARVVCLLNNFYPREAKVQWKVDNALQSGNSQESVTEQDS
KDSTYSLISTLTLSKADYEKHKVYACEVTHQGLSSPVTKSENRGECHHHHHHHH
SEQ ID NO: 2 (LC1)( HER-2)
SEQ ID NO: 4 (LC2)(EGFR)
SEQ ID NO: 10 (VH)(cMET)
Sequences for FIG. 6(B): PD-1 X CD137 X CTLA-4 TsAb_HHL
SEQ ID NO: 22 (HC1)

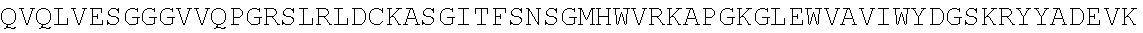

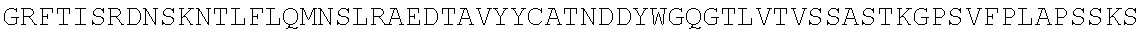

TSGGTAALGCLVKDYFPEPVTVSWNSGALTSGVATGPAVLQSSGLYSLSSVVTVPSSSLGTQTYI

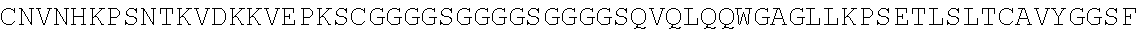

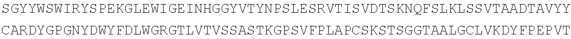

VSWNSGALTSGVHTFPAVLQSSGLYSLSSVVTVPSSSLGTQTYICNVNHKPSNTKVDKKVEPDSG

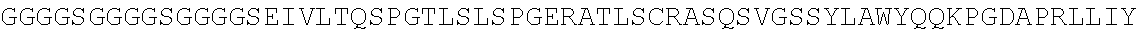

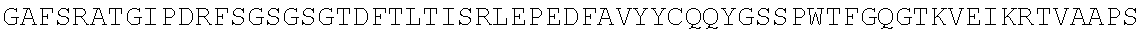

VFIFPPSDKQLKSGTARVVCLLNNFYPREAKVQWKVDNALQSGNSQESVTEQDSKDSTYSLISTL
TLSKADYEKHKVYACEVTHQGLSSPVTKSENRGECHHHHHHHH
SEQ ID NO: 6 (LC1)(PD-1)
SEQ ID NO: 8 (LC2)(CD137)
SEQ ID NO: 12 (VH)(CTLA-4)
SEQ ID NO: 23 (fragment of HC1)
WGQGTLVTVSSASTKGPSVFPLAPSSKSTSGGTAALGCLVKDYFPEPVTVSWNSGALTSGVATGP
AVLQSSGLYSLSSVVTVPSSSLGTQTYICNVNHKPSNTKVDKKVEPKSCDKTHTCPPCPAPEAAG
GPSVFLFPPKPKDTLMISRTPEVTCVVVDVSHEDPEVKENWYVDGVEVHNAKTKPREQYQSTYRV
VSVLTVLHQDWLNGKEYKCKVSNKALPAPIEKTISKAKGQPREPQVSTLPPSREEMTKNQVSLMC
LVYGFYPSDIAVEWESNGQPENNYKTTPPVLDSDGSFFLYSVLTVDKSRWQQGNVFSCSVM
SEQ ID NO: 24 ( fragment of HC2)
WGQGTLVTVSSASTKGPSVFPLAPCSKSTSGGTAALGCLVKDYFPEPVTVSWNSGALTSGVHTEP
AVLQSSGLYSLSSVVTVPSSSIGTQTYICNVNHKPSNTKVDKKVEPDSGDKTHTCPPCPAPEAAG
GPSVFLFPPKPKDTLMISRTPEVTCVVVDVSHEDPEVKENWYVDGVEVHNAKTKPREEQYQSTYR
VVSVLTVLHQDWLNGKEYKCKVSNKALPAPIEKTISKAKGQPREPQVYTLPPSRGDMTKNQVQLT
CLVKGFYPSDIAVEWESNGQPENNYKTTPPVLDSDGSFFLASKLTVDKSRWQQGNVESCSVM
SEQ ID NO: 25 (fragment of SEQ ID NO:2)
GQPKAAPSVTLFPPSSEELQANKATLVCYISDEYPGAVTVAWKADSSPVKAGVETTTPSKQSNNK
YAAWSYLSLTPEQWKSHRSYSCQVTHEGSTVEKTVAPTEC
SEQ ID NO: 26 (fragment of SEQ ID NO:4)
RTVAAPSVFIFPPSKEQLKSGTASVVCLLNNFYPREAKVQWKVDNALQSGNSQESVTEQDSKDST
YSLSSTLLLSKADYEKHKVYACEVTHQGLSSPVTKSENRGEC

1. A binding protein comprising:
a. a first heavy chain (HC1), comprising, in order from the N-terminus to the C-terminus, (i) a first heavy chain variable region (V_{H}) comprising a lysine at residue 39 and/or a glutamate at residue 62, (ii) a CH1 domain comprising an alanine at residue 172 and/or a glycine at residue 174, (iii) a CH2 domain, and (iv) a CH3 domain;
b. a first light chain (LC1), comprising, in order from the N-terminus to the C-terminus, (i) a first light chain variable region (VL) comprising an arginine at residue 1 or an aspartate at residue 38, (ii) a first light chain constant region comprising a tyrosine at residue 135 or a tryptophan at residue 176;
c. a second heavy chain (HC2), comprising, in order from the N-terminus to the C-terminus, (i) a second VH comprising a tyrosine at residue 39, (ii) a CH1 domain comprising a cysteine at residue 127, an aspartate at residue 228 or a serine at residue 230, (iii) a CH2 domain, and (iv) a CH3 domain;
d. a second light chain (LC2), comprising, in order from the N-terminus to the C-terminus, a second VL comprising a arginine at residue 38, (ii) a light chain constant region comprising a lysine at residue 122; and
e. an antigen-binding Fab.

2. A binding protein comprising:
a. a first heavy chain (HC1), comprising, in order from the N-terminus to the C-terminus, a heavy chain variable region (V_{H}) comprising a lysine at residue 39 and the amino acid sequence of SEQ ID NO: 23;
b. a first light chain (LC1), comprising, in order from the N-terminus to the C-terminus, a light chain variable region (VL) comprising i) an arginine at residue 1 and an aspartate at residue 38; and ii) the amino acid sequence of SEQ ID NO: 25; wherein said VH of HC1 and said VL of LC1 form a first antigen binding domain;
c. a second heavy chain (HC2), comprising, in order from the N-terminus to the C-terminus, i) a tyrosine at residue 39, and ii) an amino acid sequence of SEQ ID NO: 24;
d. a second light chain (LC2), comprising, in order from the N-terminus to the C-terminus, a VL comprising i) an arginine at residue 38 and ii) the amino acid sequence of SEQ ID NO: 26; wherein said VH of HC2 and said VL of LC2 form a second antigen binding domain; and
e. an antigen-binding Fab fragment.

3. The binding protein of claim 1 wherein the antigen-binding Fab comprises (i) a third VH comprising an arginine at residue 105, (ii) a CH1 domain comprising an alanine at residue 145 and a glutamate residue at 221; (iii) a VL comprising an aspartate at residue 42, and (iv) a light chain constant region comprising a lysine at residue 123 and an arginine at position 131.

4. The binding protein of claim 1 wherein i) the N-terminus of the VH of the Fab fragment is linked by a peptide linker to the C-terminus of the CH3 domain of the HC1 and/or the N-terminus of the VL of the Fab fragment is linked by a peptide linker to the C-terminus of the CH3 domain of the HC2, ii) the N-terminus of the VH of the Fab is linked by a peptide linker to the C-terminus of the CH3 domain of the HC1, (iii) the N-terminus of the VL of the Fab is linked by a peptide linker to the C-terminus of the CH3 domain of the HC2, or (iv) the C-terminus of the Fab fragment's CH1 domain or the Fab fragment's light chain C_{L}domain is linked to the N-terminus of the VH domain of the first or second V_{H};
wherein said first VH and said first VL form a first antigen binding domain, said V_{H}of HC2 and said VL of LC2 form a second antigen binding domain; and said first and second antigen binding domains and the Fab fragment specifically bind one or more target proteins.

5. The binding protein of claim 1 wherein i) the CH3 domain of the HC1 comprises a serine at residue 349, a methionine at residue 366, a tyrosine at residue 370 and a valine at residue 409; and ii) wherein the CH3 of the HC2 comprises a glycine at residue 356, a glycine at residue 357, a glutamine at residue 364 and an alanine at residue 407.

6. A binding protein comprising a polypeptide heavy chain (HC), a first polypeptide light chain (LC1), a second polypeptide light chain (LC2), and a third polypeptide light chain (LC3), in which
a. the HC has the formula, in order from the N-terminus to the C-terminus, of VH1-CH1a-L1-VH2-CH1b-L2-VH3-CH1c, wherein VH1, VH2, and VH3 are a first, second, and third heavy chain variable region, respectively, and CH1a, CH1b and CH1c are a first, second, and third IgG CH1 domains, wherein CH1a comprises an alanine and a glycine at amino acid residues 172 and 174, respectively, CH1b comprises a cysteine, an aspartate, and a serine at amino acid residues 127, 228, and 230, respectively, and CH1c comprises an alanine and a glutamate at amino acid residues 145 and 221, respectively;
b. the LC1 has the formula VL1-CLa which associates with the VH1-CH1a region of the HC to form a binding site for a first binding partner;
c. the LC2 has the formula VL2-CLb which associates with the VH2-CH1b region of the HC to form a binding site for a second binding partner;
d. the LC3 has the formula VL3-CLc which associates with the VH3-CH1c region of the HC to form a binding site for a first binding partner, wherein CLa comprises a tyrosine and a tryptophan at amino acid residues 135 and 176 respectively, CLb comprises a lysine at amino acid residue 122, and CLc comprises a lysine and an arginine at amino acid residues, 123 and 131, respectively; and
e. L1 and L2 are peptide linkers.

7. A binding protein comprising a polypeptide heavy chain (HC), a first polypeptide light chain (LC1), a second polypeptide light chain (LC2), and a third polypeptide light chain (LC3), in which the HC has the formula, in order from the N-terminus to the C-terminus, of VH1-CH1a-L1-VH2-CH1b-L2-VL3-CLc, wherein VH1 and VH2, is a first and second heavy chain variable region (V_{H}), respectively, and VL3 is a light chain variable region (VL);
a. CH1a comprises an alanine and a glycine at amino acid residues 172 and 174, respectively;
b. CH1b comprises a cysteine, an aspartate, and a serine at amino acid residues 127, 228, and 230, respectively;
c. CLc comprises a lysine and an arginine at amino acid residues, 123 and 131, respectively;
d. the LC1 has the formula VL1-CLa which associates with the VH1-CH1a region of the HC to form a binding site for a first binding partner;
e. the LC2 has the formula VL2-CLb which associates with the VH2-CHb region of the HC to form a binding site for a second binding partner;
f. the LC3 has the formula VH3-CHc which associates with the VL3-CLc region of the HC to form a binding site for a third binding partner; and
g. L1 and L2 are peptide linkers.

8. A method for producing a binding protein comprising:
a. expressing in a host cell a nucleic acid encoding the binding protein of claim 1;
b. cultivating said host cell under conditions such that said binding protein is produced; and
c. recovering from said host cell the binding protein.

9. The method according to claim 8 wherein said host cell is a mammalian cell.

10. The method according to claim 9 wherein said host cell is a HEK293 or CHO cell.

11. A binding protein produced according to claim 8.

12. A binding protein produced according to claim 9.

13. A binding protein produced according to claim 10.

14. The binding protein of claim 2 wherein the antigen-binding Fab comprises (i) a third VH comprising an arginine at residue 105, (ii) a CH1 domain comprising an alanine at residue 145 and a glutamate residue at 221; (iii) a VL comprising an aspartate at residue 42, and (iv) a light chain constant region comprising a lysine at residue 123 and an arginine at position 131.

15. The binding protein of claim 2 wherein i) the N-terminus of the VH of the Fab fragment is linked by a peptide linker to the C-terminus of the CH3 domain of the HC1 and/or the N-terminus of the VL of the Fab fragment is linked by a peptide linker to the C-terminus of the CH3 domain of the HC2, ii) the N-terminus of the VH of the Fab is linked by a peptide linker to the C-terminus of the CH3 domain of the HC1, (iii) the N-terminus of the VL of the Fab is linked by a peptide linker to the C-terminus of the CH3 domain of the HC2, or (iv) the C-terminus of the Fab fragment's CH1 domain or the Fab fragment's light chain CL domain is linked to the N-terminus of the VH domain of the first or second VH; wherein said first VH and said first VL form a first antigen binding domain, said VH of HC2 and said VL of LC2 form a second antigen binding domain; and said first and second antigen binding domains and the Fab fragment specifically bind one or more target proteins.

16. The binding protein of claim 3 wherein i) the N-terminus of the VH of the Fab fragment is linked by a peptide linker to the C-terminus of the CH3 domain of the HC1 and/or the N-terminus of the VL of the Fab fragment is linked by a peptide linker to the C-terminus of the CH3 domain of the HC2, ii) the N-terminus of the VH of the Fab is linked by a peptide linker to the C-terminus of the CH3 domain of the HC1, (iii) the N-terminus of the VL of the Fab is linked by a peptide linker to the C-terminus of the CH3 domain of the HC2, or (iv) the C-terminus of the Fab fragment's CH1 domain or the Fab fragment's light chain CL domain is linked to the N-terminus of the VH domain of the first or second VH; wherein said first VH and said first VL form a first antigen binding domain, said VH of HC2 and said VL of LC2 form a second antigen binding domain; and said first and second antigen binding domains and the Fab fragment specifically bind one or more target proteins.

17. The binding protein of claim 2 wherein i) the CH3 domain of the HC1 comprises a serine at residue 349, a methionine at residue 366, a tyrosine at residue 370 and a valine at residue 409; and ii) wherein the CH3 of the HC2 comprises a glycine at residue 356, a glycine at residue 357, a glutamine at residue 364 and an alanine at residue 407.

18. The binding protein of claim 3 wherein i) the CH3 domain of the HC1 comprises a serine at residue 349, a methionine at residue 366, a tyrosine at residue 370 and a valine at residue 409; and ii) wherein the CH3 of the HC2 comprises a glycine at residue 356, a glycine at residue 357, a glutamine at residue 364 and an alanine at residue 407.

19. The binding protein of claim 4 wherein i) the CH3 domain of the HC1 comprises a serine at residue 349, a methionine at residue 366, a tyrosine at residue 370 and a valine at residue 409; and ii) wherein the CH3 of the HC2 comprises a glycine at residue 356, a glycine at residue 357, a glutamine at residue 364 and an alanine at residue 407.

20. A method for producing a binding protein comprising:
a. expressing in a mammalian cell a nucleic acid encoding the binding protein of claim 2;
b. cultivating said host cell under conditions such that said binding protein is produced; and
c. recovering from said host cell the binding protein.

21. The method according to claim 20 wherein said host cell is a mammalian cell.

22. The method according to claim 21 wherein said host cell is a HEK293 or CHO cell.
